(19) United States
(12) Patent Application Publication
(10) Pub. No.: US 2025/0368598 A1
(43) Pub. Date: Dec. 4, 2025

(54) CATALYTIC CARBOXYCARBONYLATION OF ALKENES TO FORM ANHYDRIDES

(71) Applicants: Shrabanti BHATTACHARYA, Chapel Hill, NC (US); THE UNIVERSITY OF NORTH CAROLINA AT CHAPEL HILL, Chapel Hill, NC (US); EASTMAN CHEMICAL COMPANY, Kingsport, TN (US); North Carolina State University, Raleigh, NC (US)

(72) Inventors: Shrabanti Bhattacharya, Chapel HIll, NC (US); Mesfin Ejerssa Janka, Kingsport, TN (US); Alexander James Minden Miller, Chapel Hill, NC (US); Erik John Alexanian, Chapel Hill, NC (US); Alexander Matthew Veatch, Carrboro, NC (US); Jeffrey Alexander Bennet, Raleigh, NC (US); Drew Cunningham, Carrboro, NC (US); Malek Y.S. Ibrahim, Graz (AT); Milad Abolhasani, Apex, NC (US); Nathan Mitchell West, Kingsport, NC (US)

(21) Appl. No.: 18/876,334

(22) PCT Filed: Jun. 23, 2023
(86) PCT No.: PCT/US2023/026135

§ 371 Date: Dec. 18, 2024

Related U.S. Application Data
(60) Provisional application No. 63/355,407, filed on Jun. 24, 2022.

(51) Int. Cl. C07C 53/124; B01J 31/24

(52) U.S. Cl. CPC C07C 53/124; B01J 31/24; B01J 2531/824

(57) ABSTRACT

Efficient carbonylative synthesis of alkenes to form anhydrides using transition metal catalysts such as palladium-phosphine catalysts.

### CROSS-REFERENCE TO RELATED APPLICATIONS

This application claims priority to U.S. Provisional Application No. 63/355,407, filed Jun. 24, 2022, which is incorporated into this application by reference.

### BACKGROUND

Methods for commercially producing organic anhydrides can suffer from several disadvantages, often depending on the carbon length of the anhydride. Commercial synthesis of lower anhydrides such as acetic anhydride (C₂) can proceed efficiently either by high temperature thermal cracking of acetic acid or by high temperature, high-pressure carbonylation of an acetate using a rhodium catalyst and methyl iodide co-catalyst. On the other hand, higher (C₃₊) anhydrides are typically made from the corresponding carboxylic acid via stoichiometric use of a dehydrating agent, such as acetic anhydride or thionyl chloride. Such methods suffer from several disadvantages. For example, commercial butyric anhydride is typically made from a reactive distillation of butyric acid and acetic anhydride to produce butyric anhydride and acetic acid. This process generates two equivalents of acetic acid coproduct. In addition, butyric acid is typically made in a two-step process from propylene via hydroformylation of propylene to butyraldehyde followed by oxidation to butyric acid. Similarly, the use of thionyl chloride as a dehydrating reagent generates significant amount of HCl and SO₂ wastes. Therefore, there is a need in the industry for an atom efficient, cost effective, and safer process to make organic anhydrides.

Carbonylation is a fundamental and atom-economical functionalization of olefins that encompasses a wide scope of reactions to produce carboxylic acids, esters, aldehydes, amides, amino acids, and other derivatives in many academic and industrial settings. There has been recent interest in the synthesis of esters from the reaction of an alkene with carbon monoxide and an alcohol. However, the carbonylative synthesis of anhydrides from an alkene by reaction with carbon monoxide and a carboxylic acid has not been reported to our knowledge, likely due to the weak nucleophilic nature of carboxylic acids. There are some examples of catalytic production of propionic anhydride from ethylene. These reactions, however, use harsh conditions and are not useful for higher anhydrides. Alcohols are good nucleophiles and readily couple with metal carbonyl complexes to form esters. Conversely, carboxylic acids are poor nucleophiles, which may explain why anhydrides cannot be made by simple extension of the esterification conditions and why there are no known reports of catalyzed carboxycarbonylation of alkenes to anhydrides.

### SUMMARY

Described below is an atom-efficient technology that can involve a single-step carbonylative anhydride synthesis of alkenes to form anhydrides at mild temperatures and pressures using transition metal catalysts such as palladium-phosphine catalysts. In some embodiments, the selectivity for normal and iso isomers, as well as any mixture resulting in asymmetric normal/iso isomers, can be controlled by changing the catalyst structure or reaction conditions. C₃ or higher olefins, for example propylene or 1-heptene, can form at least two anhydride isomers. Having the ability to control the selectivity for a desired isomer by changing ligand structure or reaction conditions is advantageous for commercial use. In some embodiments, benzoyl halides and other co-catalytic additives were found to enhance catalyst solubility, activity, stability, and recyclability.

One embodiment of the method comprises contacting an ethylenically unsaturated compound with carbon monoxide and a carboxylic acid in the presence of a catalyst system obtainable by combining palladium or a palladium compound and a phosphine ligand, thereby forming the organic anhydride. In general, the method is effective not only for the reaction of separate ethylenically unsaturated compounds and carboxylic acids but also for the formation of cyclic anhydrides and poly(organic anhydrides) from compounds that include both an ethylenically unsaturated group and a carboxylic acid group, as well as from dienes and di-carboxylic acids.

### DETAILED DESCRIPTION

### A. Definitions

“Alkyl” refers to a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. “Alkyl” can be a C₁ alkyl, C₁-C₂ alkyl, C₁-C₃ alkyl, C₁-C₄ alkyl, C₁-C₅ alkyl, C₁-C₆ alkyl, C₁-C₇ alkyl, C₁-C₈ alkyl, C₁-C₉ alkyl, C₁-C₁₀ alkyl, and the like up to and including a C₁-C₂₄ alkyl. “Heteroalkyl” refers to an alkyl group in which one or more of the hydrogen atoms bonded to carbon are substituted with a heteroatom including but not limited to O, S, or N(R)₂, in which each R can independently be hydrogen or a non-hydrogen substituent.

“Cycloalkyl” refers to a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. “Heterocycloalkyl” is a non-aromatic carbon-based ring type of cycloalkyl group, where at least one of the carbon atoms of the ring is replaced with a heteroatom such as, but not limited to, nitrogen, oxygen, sulfur, or phosphorus. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol.

“Bicyclic cycloalkyl” or “bicyclic heterocycloalkyl” refers to a compound in which two or more cycloalkyl or heterocycloalkyl groups are fused together. Non-limiting examples of bicyclic cycloalkyl groups include without limitation (1r,4r)-bicyclo[2.1.1]hexane, (1s,4s)-bicyclo[2.2.1]heptane, (1R,6S)-bicyclo[4.2.0]octane, adamantane, and the like. Non-limiting examples of bicyclic heterocycloalkyl groups include without limitation any of the foregoing groups in which at least one of the carbon atoms is replaced with a heteroatom such as nitrogen, oxygen, sulfur, or phosphorus.

“Alkenyl” refers to a hydrocarbon having from 2 to 24 carbons with a structural formula containing at least one carbon-carbon double bond. Asymmetric structures such as (A¹A²)C═C(A³ A⁴) are intended to include both the E and Z isomers. The alkenyl group can be substituted with one or more groups including alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, among others.

“Cycloalkenyl” refers to a non-aromatic carbon-based ring composed of at least three carbon atoms and containing at least one carbon-carbon double bound, i.e., C═C. Examples of cycloalkenyl groups include cyclopropenyl, cyclobutenyl, cyclopentenyl, cyclopentadienyl, cyclohexenyl, cyclohexadienyl, norbornenyl, among others. The term “heterocycloalkenyl” is a type of cycloalkenyl group and is included within the meaning of the term “cycloalkenyl,” where at least one of the carbon atoms of the ring is replaced with a heteroatom such as nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, among others.

“Alkynyl” means a hydrocarbon group of 2 to 24 carbon atoms with a structural formula containing at least one carbon-carbon triple bond. The alkynyl group can be unsubstituted or substituted with one or more groups including alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, among others.

“Cycloalkynyl” refers to a non-aromatic carbon-based ring composed of at least seven carbon atoms and containing at least one carbon-carbon triple bound. Examples of cycloalkynyl groups include cycloheptynyl, cyclooctynyl, cyclononynyl, and the like. The term “heterocycloalkynyl” is a type of cycloalkenyl group and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, among others.

“Aryl” refers to a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, −NH₂, carboxylic acid, ester, ether, halide, hydroxy, ketone, azide, nitro, silyl, sulfo-oxo, or thiol as described herein. In addition, the aryl group can be a single ring structure or comprise multiple ring structures that are either fused ring structures or attached via one or more bridging groups such as a carbon-carbon bond. For example, aryl can include biaryl in which two aryl groups are bound together via a fused ring structure, as in naphthalene, or are attached via one or more carbon-carbon bonds, as in biphenyl.

“Heteroaryl” refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further non-limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

“Halide” refers to F, Cl, Br, or I. “Haloalkyl,” “haloalkenyl,” and the like refer to compounds or groups which include at least one halide substituent at any position.

“Ferrocenyl” refers to any functional group that includes the ferrocene structure below (substituted or unsubstituted at any position):

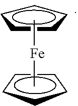

“Oxydibenzyl” refers to any functional group that includes the structure below (substituted or unsubstituted at any position):

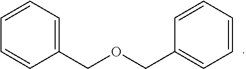 [SMILES: c1ccc(COCc2ccccc2)cc1]

“Quinolinyl” refers to any functional group that includes the structure below (substituted or unsubstited at any position):

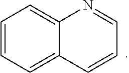

“Acridinyl” refers to any functional group that includes the structure below (substituted or unsubstituted at any position):

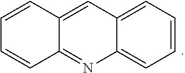 [SMILES: c1ccc2nc3ccccc3cc2c1]

“Dihydroacridinyl” refers to any functional group that includes the structure below (substituted or unsubstituted at any position):

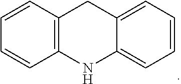 [SMILES: c1ccc2c(c1)Cc1ccccc1N2]

“Xanthenyl” refers to any functional group that includes the structure below (substituted or unsubstituted at any position):

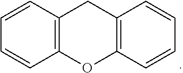 [SMILES: c1ccc2c(c1)Cc1ccccc1O2]

“10H-phenoxazinyl” refers to any functional group that includes the structure below (substituted or unsubstituted at any position):

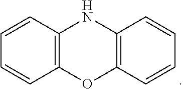 [SMILES: c1ccc2c(c1)Nc1ccccc1O2]

“Reactor” means any suitable vessel useful for performing the catalytic reaction methods. The reactor can be a smaller, lab-scale reactor, or a larger commercial scale reactor. Smaller reactors include, without limitation, steel pressure reactors containing glass or TEFLON (PTFE) liners. In other aspects, the reactor can be a Hastelloy autoclave having a suitable volume. In some aspects, the reactor can be equipped with an infrared spectroscopy probe for in situ monitoring of the reaction mixture.

“Molar ratio” refers to the moles of one substance relative to the moles of another substance.

“Turnover number” or “TON” refers to the moles of a reaction product divided by the moles of a precatalyst or catalyst added to or formed within the reactor.

“Partial pressure” refers to the pressure of a constituent gas in the atmosphere of the reaction medium, which is the notional pressure of that constituent gas if the gas occupied the entire volume of the original mixture at the same temperature.

When the term “about” precedes a numerical value, the numerical value can vary within ±10% unless specified otherwise.

### B. Catalytic Carboxycarbonylation Method

The catalytic method generally comprises contacting an ethylenically unsaturated compound with carbon monoxide and a carboxylic acid in the presence of a catalyst system obtainable by combining palladium or a palladium compound and a phosphine ligand, thereby forming the organic anhydride. In addition to the general reaction shown below in Scheme 1, the method is also useful for forming cyclic organic anhydrides and poly(organic anhydrides).

The catalyst system can generally obtained by combining palladium or a palladium compound and a phosphine ligand, which creates a catalytic palladium-phosphine complex. Formation of the catalyst system can occur prior to the reaction or can occur in situ, e.g., a reactor can be charged with the starting materials and the palladium or palladium compound and the phosphine ligand. In one aspect, the palladium compound is a palladium(0) or palladium(II) compound. Specific examples of palladium compounds include without limitation tris(dibenzylideneacetone)dipalladium(0), palladium(π-cinnamyl) chloride dimer, Pd(OAc)₂, PdCl₂, Pd(PhCN)₂Cl₂, Pd(MeCN)₂Cl₂, Pd(PPh₃)₂Cl₂, Pd(COD)Cl₂, or [Pd(π-allyl)Cl]₂. In a further specific aspect, the palladium compound is PdCl₂, Pd(PhCN)₂Cl₂, Pd(MeCN)₂Cl₂, Pd(PPh₃)₂Cl₂, Pd(COD)Cl₂, [Pd(π-allyl)Cl]₂, or [Pd(cinnamyl)Cl]₂.

Other reaction conditions will generally vary depending on scale and other parameters. A variety of temperatures can be used. In one aspect, the reaction is carried out at a temperature of at least 50° C., e.g., 50-200° C., or 50-130° C. In a further aspect, the reaction is carried out at a temperature of at least 70° C., e.g., 70-200° C., or 70-130° C. In a further aspect, the reaction is carried out at a temperature of at least 100° C., e.g., 100-130° C.

The reaction can be carried out under light irradiation. A variety of wavelengths of light can be used. In one aspect, the reaction is carried out under irradiation from a light source wherein the light source has a wavelength of at least 300 nm, e.g., 300-500 nm, or 300-430 nm. In a further aspect, the reaction is carried out under irradiation from a light source wherein the light source has a wavelength of at least 350 nm, e.g., 350-500 nm, or 350-430 nm. In a further aspect, the reaction is carried out under irradiation from a light source wherein the light source has a wavelength ranging from 350-390 nm.

The reaction can generally be carried out at a suitable time which can depend on a variety of factors. Reaction products, however, can be monitored to determine when the reaction mixture should be quenched if necessary. Suitable reaction times include for example 3-24 hours, e.g., 10-15 hours, or much longer times when carried out on large industrial scales. In general, the reaction can be carried out for any suitable time as indicated by methods for measuring reaction progress and completion. In addition, the carbonylation reaction can be implemented as part of a batch or continuous process.

The atmosphere in which the catalytic carboxycarbonylation is carried out includes carbon monoxide or a source thereof. In one aspect, the carbon monoxide can be present in a syngas composition comprising hydrogen gas. In addition, any suitable source of carbon monoxide gas can be used, including precursor materials that can form carbon monoxide in a reactor, for example under increased pressure. Examples of precursor materials that can form carbon monoxide in situ include carbon dioxide, metal carbonyls, formic acid derivatives, and methanol, among others. These sources of carbon monoxide can be desirable for minimizing any toxicity and transportation problems resulting from gaseous carbon monoxide.

The partial pressure of the carbon monoxide in the reactor can vary. In one aspect, the partial pressure of carbon monoxide is at least 1 atmospheric pressure (atm). In a further aspect, the partial pressure of carbon monoxide ranges from about 1 atmospheric pressure (atm) to about 100 atm. In a further aspect, higher pressures of carbon monoxide can be used, e.g., 10-100 atm, such as at least 20 atm, at least 30 atm, and at about 40 atm of carbon monoxide. In some aspects, the carbon monoxide or source thereof, or reactor, is substantially free of water, or in some aspects, free of water.

The catalytic reaction can be carried out neat, or in some aspects in a suitable solvent. In one aspect, the reaction is carried out neat, i.e., the reaction medium consists essentially of or in some aspects consists of the ethylenically unsaturated compound, the carboxylic acid, and the catalyst system (optionally including a co-catalytic additive) under an atmosphere that at least partially comprises carbon monoxide or a source thereof.

In another aspect, the reaction can be carried out in a solvent. In one aspect, the solvent is aromatic. In a further aspect, the solvent is a halogenated, nitrile, or ethereal solvent. Non-limiting specific examples of suitable solvents include acetonitrile, chlorobenzene, dichloromethane, dichloroethane, trifluorotoluene, perfluorotoluene, tetrachloroethane, tetrahydrofuran, benzonitrile, chlorobenzene, pyridine, dibenzyl ether, xylene, toluene, methyl acetate, methyl propionate, ethyl acetate, propyl acetate, butyl acetate, isobutyl acetate, dimethylformamide, and dimethyl sulfoxide.

In some aspects, the reaction medium can further comprise a co-catalytic additive. In one aspect, the co-catalytic additive is an acid. In some aspects, the acid can be an organic acid. In a further aspect, the co-catalytic additive is an acyl electrophile. Non-limiting examples include trifluoroacetic anhydride or acetic anhydride. In another aspect, the co-catalytic additive is halogenated. In a further specific aspect, the co-catalytic additive is an aryl halide or benzoyl halide.

Specific non-limiting examples of co-catalytic additives include cinnamyl chloride, tetrabutylammonium chloride (TBACl), tetrabutylammonium bromide (TBABr), tetrabutylammonium iodide (TBAI), p-toluenesulfonic acid (PTSA), benzyl chloride, benzoyl bromide, cesium iodide, methyl iodide, 4-iodobenzotrifluoride, acyl chloride, lithium chloride, lithium bromide, lithium iodide, 1-iodooctane, a combination of benzyl chloride and lithium chloride, acetic anhydride, trifluoroacetic acid (TFA), trifluoroacetic anhydride, hydrochloric acid (HCl), HCl in a solvent such as dioxane, benzenesulfonic acid (PhSO₃H), methanesulfonic acid (MeSO₃H), and any combination thereof.

### 1. Ethylenically Unsaturated Compounds

The ethylenically unsaturated substrate can vary. As discussed above, for cyclic organic anhydrides, a single compound can have an ethylenically unsaturated group, e.g., a terminal alkene, in addition to a carboxylic acid functional group, which can afford the corresponding organic anhydride(s). Similarly, the ethylenically unsaturated compound can be a diene, e.g., a di-terminal alkene, which can react with a di-carboxylic acid such as a di-terminal carboxylic acid, to afford the corresponding poly(organic anhydride).

For other instances in which the ethylenically unsaturated compound and carboxylic acid are individual small molecules, the ethylenically unsaturated compound will generally be a monosubstituted, disubstituted, or trisubstituted alkene. In one aspect, the ethylenically unsaturated compound is a terminal alkene.

In a further aspect, the ethylenically unsaturated compound has the formula (I):

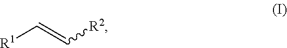

wherein R¹ and R² are independently hydrogen, halide, C₁-C₂₄ alkyl, C₁-C₂₄ heteroalkyl, C₁-C₂₄ alkenyl, C₁-C₂₄ alkynyl, C₁-C₂₄ haloalkyl, C₁-C₂₄ haloalkenyl, cycloalkyl, heterocycloalkyl, bicyclic cycloalkyl, bicyclic heterocycloalkyl, cycloalkenyl, cycloalkynyl, aryl, or heteroaryl; or wherein R¹ and R² together form a ring having 4 to 10 carbons; and wherein the wavy bond denotes any geometric isomer.

In one aspect, the ethylenically unsaturated compound has the formula (I), R² is hydrogen (i.e., the ethylenically unsaturated compound is a terminal alkene), and R¹ is hydrogen, halide, C₁-C₂₄ alkyl, C₁-C₂₄ heteroalkyl, cycloalkyl, heterocycloalkyl, bicyclic cycloalkyl, or bicyclic heterocycloalkyl. In a further aspect, the ethylenically unsaturated compound has the formula (I), R² is hydrogen, and R¹ is hydrogen, halide, C₁-C₂₀ alkyl, C₁-C₂₀ heteroalkyl, cycloalkyl, heterocycloalkyl, bicyclic cycloalkyl, or bicyclic heterocycloalkyl. In a further aspect, the ethylenically unsaturated compound has the formula (I), R² is hydrogen, and R¹ is hydrogen, halide, C₁-C₁₈ alkyl, C₁-C₁₈ heteroalkyl, cycloalkyl, heterocycloalkyl, bicyclic cycloalkyl, or bicyclic heterocycloalkyl. In a further aspect, the ethylenically unsaturated compound has the formula (I), R² is hydrogen, and R¹ is hydrogen, halide, C₁-C₁₆ alkyl, C₁-C₁₆ heteroalkyl, cycloalkyl, heterocycloalkyl, bicyclic cycloalkyl, or bicyclic heterocycloalkyl. In a further aspect, the ethylenically unsaturated compound has the formula (I), R² is hydrogen, and R¹ is hydrogen, halide, C₁-C₁₄ alkyl, C₁-C₁₄ heteroalkyl, cycloalkyl, heterocycloalkyl, bicyclic cycloalkyl, or bicyclic heterocycloalkyl. In a further aspect, the ethylenically unsaturated compound has the formula (I), R² is hydrogen, and R¹ is hydrogen, halide, C₁-C₁₂ alkyl, C₁-C₁₂ heteroalkyl, cycloalkyl, heterocycloalkyl, bicyclic cycloalkyl, or bicyclic heterocycloalkyl. In a further aspect, the ethylenically unsaturated compound has the formula (I), R² is hydrogen, and R¹ is hydrogen, halide, C₁-C₁₀ alkyl, C₁-C₁₀ heteroalkyl, cycloalkyl, heterocycloalkyl, bicyclic cycloalkyl, or bicyclic heterocycloalkyl. In a further aspect, the ethylenically unsaturated compound has the formula (I), R² is hydrogen, and R¹ is hydrogen, halide, C₁-C₈ alkyl, C₁-C₈ heteroalkyl, cycloalkyl, heterocycloalkyl, bicyclic cycloalkyl, or bicyclic heterocycloalkyl. Specific non-limiting examples of suitable ethylenically unsaturated compounds include ethylene, propylene, and 1-heptene.

### 2. Carboxylic Acids

The carboxylic acid can be added to the reaction mixture or formed in situ from a variety of suitable precursors. In one aspect, the carboxylic acid has the formula (II):

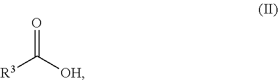

wherein R³ is C₁-C₂₄ alkyl, C₁-C₂₄ alkenyl, C₁-C₂₄ alkynyl, C₁-C₂₄ haloalkyl, C₁-C₂₄ haloalkenyl, C₁-C₂₄ haloalkynyl, cycloalkyl, heterocycloalkyl, bicyclic cycloalkyl, bicycylic heterocycloalkyl, cycloalkenyl, cycloalkynyl, aryl, or heteroaryl.

In a further aspect, the carboxylic acid has the formula (II); wherein R³ is C₁-C₂₄ alkyl, C₁-C₂₄ haloalkyl, cycloalkyl, heterocycloalkyl, bicyclic cycloalkyl, bicycylic heterocycloalkyl, cycloalkenyl, cycloalkynyl, aryl, or heteroaryl. In a further aspect, the carboxylic acid has the formula (II); wherein R³ is C₁-C₂₀ alkyl, C₁-C₂₀ haloalkyl, cycloalkyl, heterocycloalkyl, bicyclic cycloalkyl, bicycylic heterocycloalkyl, cycloalkenyl, cycloalkynyl, aryl, or heteroaryl. In a further aspect, the carboxylic acid has the formula (II); wherein R³ is C₁-C₁₈ alkyl, C₁-C₁₈ haloalkyl, cycloalkyl, heterocycloalkyl, bicyclic cycloalkyl, bicycylic heterocycloalkyl, cycloalkenyl, cycloalkynyl, aryl, or heteroaryl. In a further aspect, the carboxylic acid has the formula (II); wherein R³ is C₁-C₁₆ alkyl, C₁-C₁₆ haloalkyl, cycloalkyl, heterocycloalkyl, bicyclic cycloalkyl, bicycylic heterocycloalkyl, cycloalkenyl, cycloalkynyl, aryl, or heteroaryl. In a further aspect, the carboxylic acid has the formula (II); wherein R³ is C₁-C₁₄ alkyl, C₁-C₁₄ haloalkyl, cycloalkyl, heterocycloalkyl, bicyclic cycloalkyl, bicycylic heterocycloalkyl, cycloalkenyl, cycloalkynyl, aryl, or heteroaryl. In a further aspect, the carboxylic acid has the formula (II); wherein R³ is C₁-C₁₂ alkyl, C₁-C₁₂ haloalkyl, cycloalkyl, heterocycloalkyl, bicyclic cycloalkyl, bicycylic heterocycloalkyl, cycloalkenyl, cycloalkynyl, aryl, or heteroaryl. In a further aspect, the carboxylic acid has the formula (II); wherein R³ is C₁-C₁₀ alkyl, C₁-C₁₀ haloalkyl, cycloalkyl, heterocycloalkyl, bicyclic cycloalkyl, bicycylic heterocycloalkyl, cycloalkenyl, cycloalkynyl, aryl, or heteroaryl. In a further aspect, the carboxylic acid has the formula (II); wherein R³ is C₁-C₈ alkyl, C₁-C₈ haloalkyl, cycloalkyl, heterocycloalkyl, bicyclic cycloalkyl, bicycylic heterocycloalkyl, cycloalkenyl, cycloalkynyl, aryl, or heteroaryl.

In one aspect, the carboxylic acid has only one carbon atom more than the ethylenically unsaturated compound. Symmetrical anhydrides can be prepared with the carboxylic acid and the ethylenically unsaturated compound are so matched.

In a specific aspect, the ethylenically unsaturated compound is propylene, the carboxylic acid is isobutyric acid, and the organic anhydride is isobutyric anhydride. This reaction will generally proceed according to Scheme 2 (where the co-catalytic additive and solvent are independently optional). The reaction product here and elsewhere will typically result in the desired organic anhydride as well as isomers of the organic anhydride, as shown in the Scheme 1 below.

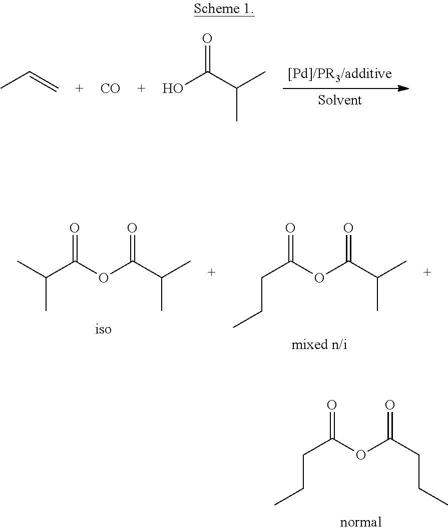

This specific aspect illustrates the versatility and efficiency of the disclosed catalytic method. Scheme 2, for example, compares the disclosed method with an existing method for preparing isobutyric anhydride (IBAN), which can be useful in preparing a common polyester precursor, 2,2,4,4-tetramethyl-1,3-cyclobutanediol (TMCD).

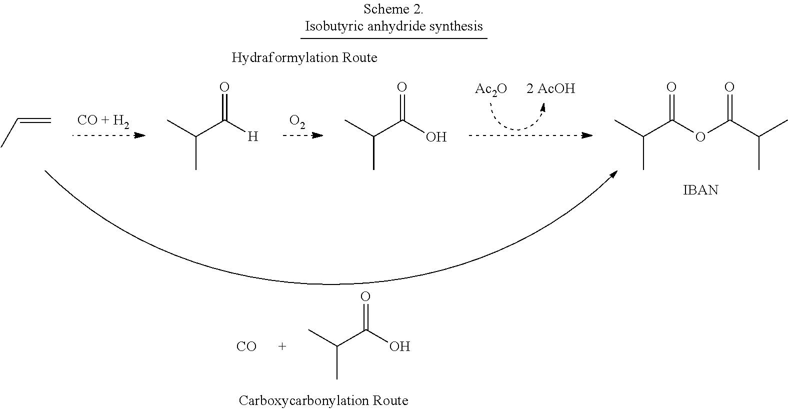

2,2,4,4-tetramethyl-1,3-cyclobutanediol Synthesis from Isobutryic Anhydride

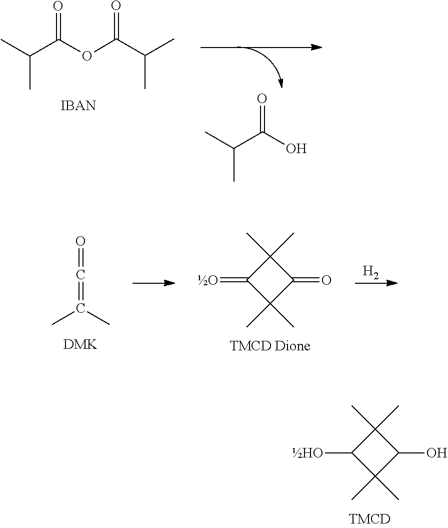

Thus, in one specific industrial aspect of the method, the method can further comprise cracking the isobutyric anhydride formed through carboxycarbonylation to generate dimethylketene, dimerizing two equivalents of the dimethylketene to afford 2,2,4,4-tetramethyl-1,3-cyclobutanedione, and hydrogenating the 2,2,4,4-tetramethyl-1,3-cyclobutanedione to form 2,2,4,4-tetramethyl-1,3-cyclobutanediol. In a further aspect, the method can further comprise polymerizing the 2,2,4,4-tetramethyl-1,3-cyclobutanediol, optionally with one or more comonomers, to form a polyester. In a further aspect, the isobutyric acid by-product from dimethylketene generation can be re-used in a subsequent carboxycarbonylation reaction.

### 3. Phosphine Ligands

A variety of phosphine ligands can be used to obtain the palladium-phosphine catalyst for the carboxycarbonylation reaction. In one aspect, the phosphine ligand is monodentate or bidentate. In another aspect, the phosphine ligand has the formula (III) or (IV):

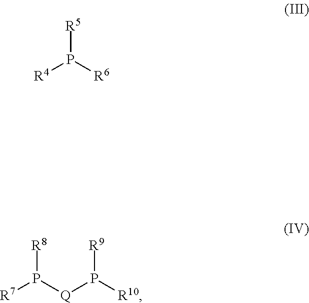

wherein R⁴-R¹⁰ are independently halide, C₁-C₂₄ alkyl, C₂-C₂₄ alkenyl, C₂-C₂₄ alkynyl, C₁-C₂₄ haloalkyl, C₂-C₂₄ haloalkenyl, C₂-C₂₀ haloalkynyl, cycloalkyl, heterocycloalkyl, bicyclic cycloalkyl, bicycylic heterocycloalkyl, cycloalkenyl, cycloalkynyl, aryl, heteroaryl, ferrocenyl, or OR¹¹, wherein R¹¹ is halide, C₁-C₂₄ alkyl, C₂-C₂₄ alkenyl, C₂-C₂₄ alkynyl, C₁-C₂₄ haloalkyl, C₂-C₂₄ haloalkenyl, C₂-C₂₄ haloalkynyl, cycloalkyl, heterocycloalkyl, bicyclic cycloalkyl, bicycylic heterocycloalkyl, cycloalkenyl, cycloalkynyl, aryl, heteroaryl, or ferrocenyl; and wherein Q is C₁-C₆ alkyl, C₂-C₆ alkenyl, C₂-C₆ alkynyl, aryl, heteroaryl, heteroarenyl, oxydibenzyl, ferrocenyl, quinolinyl, acridinyl, dihydroacridinyl, xanthenyl, or 10H-phenoxazinyl.

In a further aspect, the phosphine ligand has the formula (IV); wherein R⁷-R¹⁰ are independently cycloalkyl, heterocycloalkyl, bicyclic cycloalkyl, bicycylic heterocycloalkyl, cycloalkenyl, cycloalkynyl, aryl, heteroaryl, or ferrocenyl; and wherein Q is aryl, heteroaryl, heteroarenyl, oxydibenzyl, ferrocenyl, quinolinyl, acridinyl, dihydroacridinyl, xanthenyl, or 10H-phenoxazinyl.

In a further aspect, the phosphine ligand has the formula (IV), wherein R⁷-R¹⁰ are independently aryl, and Q is aryl, heteroaryl, heteroarenyl, oxydibenzyl, ferrocenyl, quinolinyl, acridinyl, dihydroacridinyl, xanthenyl, or 10H-phenoxazinyl.

In another aspect, the phosphine ligand has the formula (V):

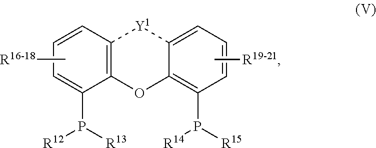

wherein R¹²-R¹⁵ are independently aryl, C₁-C₂₄ alkyl, 2-pyridyl, or 2-furyl; R¹⁶-R²¹ are independently hydrogen or C₁-C₂₄ alkyl; wherein the broken bond lines indicate optional bonds; wherein Y¹ if present is C(CH₃)₂, NH, NCH₃, or CH₂.

In another aspect, the phosphine ligand has the formula (VI):

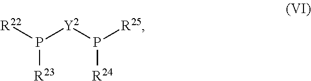

wherein R²²-R²⁵ are independently aryl, C₁-C₂₄ alkyl, 2-pyridyl, or 2-furyl, and wherein Y² is CH₂, C₂H₄, C₃H₆, or C₄H₈.

In another aspect, the phosphine ligand has the formula (VII):

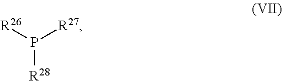

wherein R²⁶-R²⁸ are independently aryl, C₁-C₂₄ alkyl, 2-pyridyl, or 2-furyl.

In another aspect, the phosphine ligand has the formula (VIII):

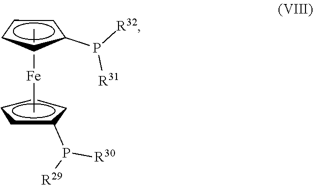

wherein R²⁹-R³² are independently aryl, C₁-C₂₄ alkyl, 2-pyridyl, or 2-furyl.

In another aspect, the phosphine ligand has the formula (IX):

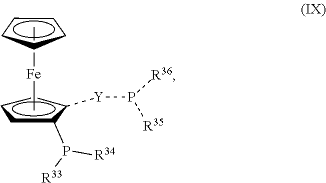

wherein R³³, R³⁴, and R³⁵-R³⁶ (when present), are independently aryl, C₁-C₂₄ alkyl, 2-pyridyl, or 2-furyl; wherein Y if present is CHCH₃, CH₂, or CHR³⁷, wherein R³⁷ is C₁-C₂₄ alkyl; wherein the broken bond lines indicate optional bonds, i.e., that the —YPR³⁵R³⁶ is an optional substituent.

Specific, non-limiting examples of phosphine ligands include the following:

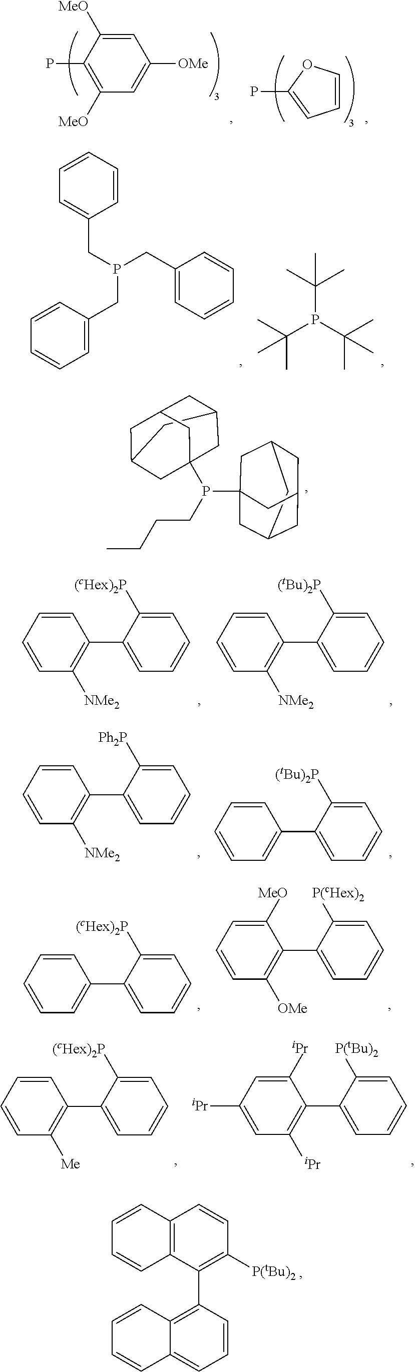

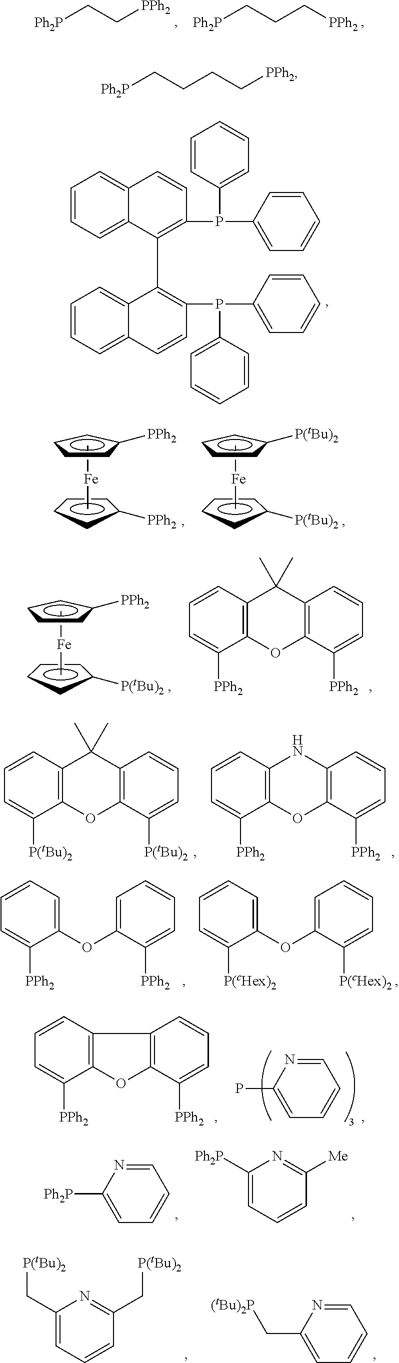

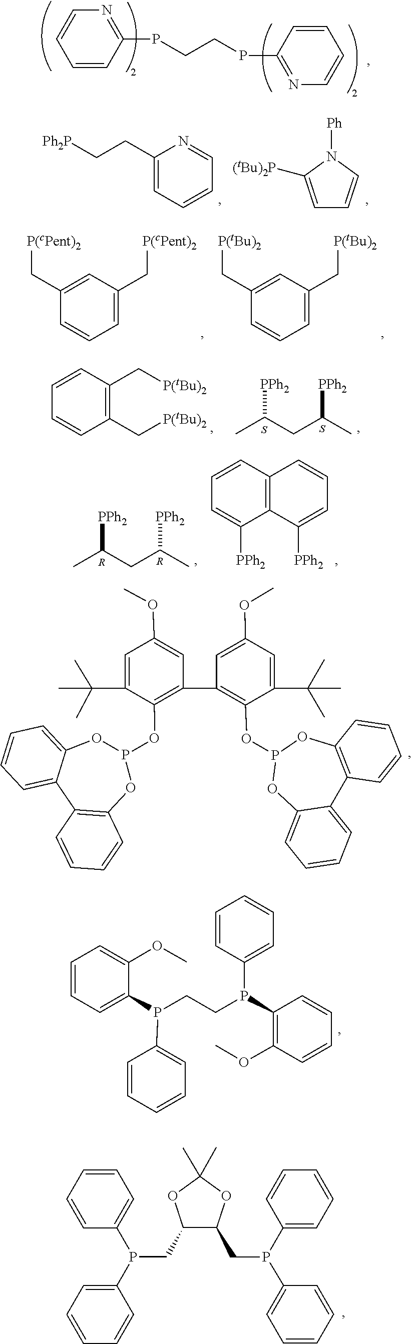

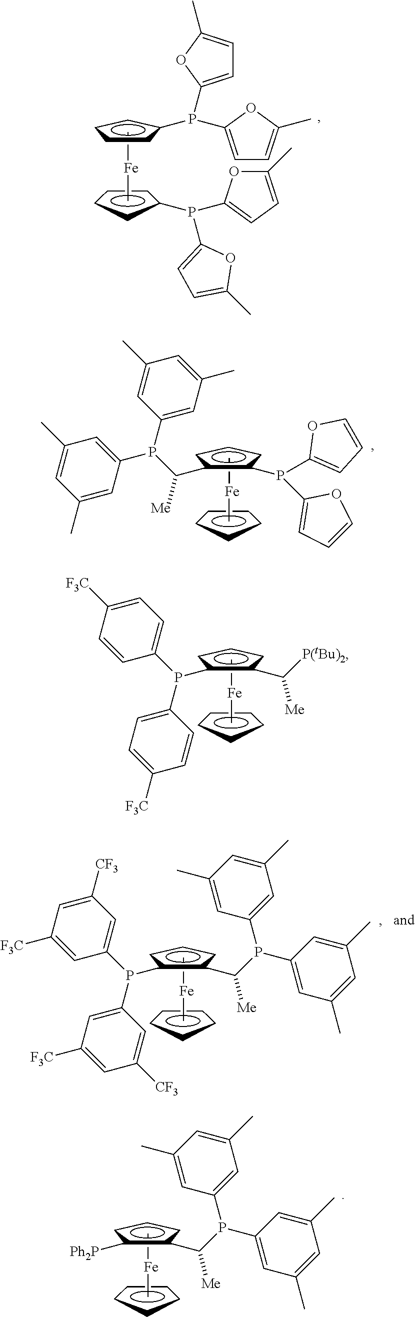

Other specific examples of phosphine ligands include triphenylphosphine, xphos, trioctylphosphine, triethyl phosphite, (9,9-Dimethyl-9H-xanthene-4,5-diyl)bis(diphenylphosphane) (xantphos), 2-chloro-4,4,5,5-tetramethyl-1,3,2-dioxaphospholane, tri-tert-butylphosphine, dppf, triphenylphosphine oxide, tricyclohexylphosphine, trioctylphosphine, diphenylphosphine, rac-BINAP, tris(dimethylamino)phosphine, tri-tert-butylphosphonium tetrafluoroborate, trioctylphosphine oxide, tri(o-tolyl)phosphine, tributylphosphine, triphenyl phosphite, tBuXPhos, tri-n-butylphosphine, chlorodiphenylphosphine, 1,3-bis(diphenylphosphino)propane, ethylenebis(diphenylphosphine), di-tert-butylchlorophosphine, 1,4-bis(diphenylphosphino)butane, trimethylphosphine, chlorodiisopropylphosphine, 1,2-bis(dicyclohexylphosphino)ethane, tris(diethylamino)phosphine, 1,2-bis(diphenylphosphino benzene, tricyclohexylphosphine tetrafluoroborate, diphenylphosphinic chloride, trimethyl phosphite, (oxydi-2,1-phenylene)bis(diphenylphosphine), tri(2 furyl)phosphine, triphenylphosphine, diphenylphosphine oxide, 1,2,3,4,5-pentaphenyl-1′-(di-tert-butylphosphino)ferrocene, tributylphosphine, trioctylphosphine oxide, dimethylphenylphosphine, DTBPF, phenylphosphonic dichloride, chlorodicyclohexylphosphine, p,p-dichlorophenylphosphine, methylphosphonic dichloride, 1,3,5-triaza-7-phosphaadamantane, me4tbutylxphos, tris(trimethylsilyl)phosphine, tri-n-butylphosphine, diethyl methylphosphonite, tributylphosphine oxide, triethylphosphine, triethylphosphine oxide, di-tert-butyl phosphite, bis(diphenylphosphino)methane, tris(hydroxymethyl)phosphine, tris(4-methoxyphenyl)phosphine, 1,2-bis(dimethylphosphino)ethane, di-tert-butyl(methyl)phosphonium tetrafluoroborate, 2-(diphenylphosphino)ethylamine, methyldiphenylphosphine, di-tert-butylphosphine, N-XantPhos, tri(p-tolyl)phosphine, trimethyl phosphite, tert-butyldichlorophosphine, tris(4-fluorophenyl)phosphine, 2-chloro-1,3,2-benzodioxaphosphorin-4-one, triphenylphosphine hydrobromide, 5-(di-tert-butylphosphino)-1′, 3′, 5′-triphenyl-1′H-┌1,4′┐bipyrazole, tris(2,4,6-trimethylphenyl)phosphine, 4-(diphenylphosphino)styrene, tris(pentafluorophenyl)phosphine, diphenyl-2-pyridylphosphine, 1,3,5,7-tetramethyl-6-phenyl-2,4,8-trioxa-6-phosphaadamantane, tris(o-methoxyphenyl)phosphine, dicyclohexylphosphine, bis(diethylamino)chlorophosphine, diisopropylphosphoramidous dichloride, tris(4-trifluoromethylphenyl)phosphine, 4-(Diphenylphosphino)benzoic acid, di-1-adamantylphosphine, 1,1′-bis(diisopropylphosphino)ferrocene, 1,1′-binaphthyl-2,2′-diyl hydrogenphosphate, 2-(di-tert-butylphosphino)-1-phenylindole, 1,5-bis(diphenylphosphino)pentane, MePhos, 1,1,1-tris(diphenylphosphinomethyl)ethane, di(1-adamantyl)-n-butylphosphine hydriodide, 2-(diphenylphosphino)benzaldehyde, di-tert-butyl n,n-diisopropylphosphoramidite, (6-aminohexyl)triphenylphosphonium bromide hydrobromide, cis-1,2-bis(diphenylphosphino)ethylene, bis(2-diphenylphosphinoethyl)phenylphosphine, 1,1′-bis(dicyclohexylphosphino)ferrocene, 1,3-bis(dicyclohexylphosphino)propane bis(tetrafluoroborate), cyclohexyldiphenylphosphine, diethylphosphoramidous dichloride, tris(1-pyrrolidinyl)phosphine, tris(2,4,6-trimethoxyphenyl)phosphine, tBuXPhos, diphosphoryl chloride, P-chlorodiphenylphosphine, tBuMePhos, 1,3-Bis(dicyclohexylphosphino)propane, 2-(diphenylphosphino)benzoic acid, ethylenebis(diphenylphosphine), dimethyl phenylphosphonite, chloro(tert-butyl)phenylphosphine, diphenylvinylphosphine, 6,6′-[(3,3′-Di-tert-butyl-5,5′-dimethoxy-1,1′-biphenyl-2,2′-diyl)bis(oxy)]bis(dibenzo[d,f][1,3,2]dioxaphosphepin), (2-hydroxyphenyl)diphenylphosphine, di(1-adamantyl)-2-dimethylaminophenylphosphine, tris[3,5-bis(trifluoromethyl)phenyl]phosphine, 2,6-bis(di-tert-butylphosphinomethyl)pyridine, triisopropylphosphine, chlorodiethylphosphine, cyclohexyldichlorophosphine, diethylphosphine, ethyl diphenylphosphinite, dicyclohexyl(4-(N,N-dimethylamino)phenyl)phosphine, 3-Methyl-1-phenyl-2-phospholene 1-oxide, di(1-adamantyl)chlorophosphine, tris[2-(diphenylphosphino)ethyl]phosphine, 4-chlorophenyl dichlorophosphate, tripropylphosphine, di-tert-butylphosphine oxide, 1,3-bis(di-tert-butylphosphinomethyl)benzene, bis(dicyclohexylphosphino)methane, diphenyl(2-methoxyphenyl)phosphine, 1,2-bis(di-tert-butylphosphinomethyl)benzene, N,N-diisopropylmethylphosphonamidic chloride, ethyldiphenylphosphine, methyl N,N,N′,N′-tetraisopropylphosphorodiamidite, bis(dimethylamino)chlorophosphine, di(1-adamantyl)benzylphosphine, tris(4-chlorophenyl)phosphine, diethylphenylphosphine, bis(diphenylphosphino)acetylene, 1,2-bis(dichlorophosphino)ethane, tri-1-naphthylphosphine, 2-(di-tert-butyl-phosphino)-1-phenyl-1h-pyrrole, 4-(dimethylamino)phenyldiphenylphosphine, allyldiphenylphosphine, o-phenylene phosphorochloridate, bis(dicyclohexylphosphinophenyl) ether, methyldiphenylphosphine oxide, dicyclohexylphenylphosphine, tetrapropylphosphonium bromide, 2-[2-(dicyclohexylphosphino)phenyl]-N-methylindole, di(o-tolyl)phosphine, 9,9-dimethyl-4,5-bis(di-tert-butylphosphino)xanthene, (6-bromohexyl)triphenylphosphonium bromide, (RS)-1-(2-diphenylphosphino-1-naphthyl)isoquinoline, o-phenylene phosphorochloridite, trimethyl phosphite, dichloroisopropylphosphine, diphenyl(trimethylsilyl)phosphine, bis(2,4,6-trimethylphenyl)phosphine, 1-diphenylphosphino-1′-(di-tert-butylphosphino)ferrocene, 1,2-bis(diphenylphosphino)ethane monooxide, bis(3,5-di(trifluoromethyl)phenyl)phosphine, (2-bromophenyl)diphenylphosphine, tributylphosphine tetrafluoroborate, Tris(3,5-dimethylphenyl)phosphine, tert-butyldiphenylphosphine, 2-(dicyclohexylphosphino)-1-phenyl-1H-pyrrole, 1,6-bis(diphenylphosphino)hexane, trioctylphosphine oxide, 2-(2-(diphenylphosphino)ethyl)pyridine, trans-1,2-bis(diphenylphosphino)ethylene, bis(4-methoxyphenyl)chlorophosphine, tris(4-methoxy-3,5-dimethylphenyl)phosphine, bis(dimethylphosphino)methane, bis(2,4,6-trimethylphenyl)phosphorus chloride, (4-hydroxyphenyl)diphenylphosphine, bis(3,5-bis(trifluoromethyl)phenyl)(2′6′-bis(dimethylamino)-3,6-dimethoxybiphenyl-2-yl)phosphine, P,P′-(9,9-dimethyl-9H-xanthene-4,5-diyl)bis[N,N,N′,N′-tetraethyl-phosphonous diamide], (R)-(4,4′,6,6′-tetramethoxybiphenyl-2,2′-diyl)bis(bis(3,5-di-tert-butyl-4-methoxyphenyl)phosphine, diphenyl(p-tolyl)phosphine, (R)-(4,4′,6,6′-tetramethoxybiphenyl-2,2′-diyl)bis(bis(3,5-dimethylphenyl)phosphine), 4-(diethylphosphino)-N,N-dimethylaniline, bis(3,5-dimethylphenyl)chlorophosphine, bis(diethylamino)phenylphosphine, 5,5-dimethyl-1,3,2-dioxaphosphorinan-2-one, di-tert-butylphenylphosphine, 2-((di-tert-butylphosphinomethyl)-6-diethylaminomethyl)pyridine, bis(dichlorophosphino)methane, bis(3,5-dimethylphenyl)phosphine, 3-(diphenylphosphino)-1-propylamine, 1,4-bis(dicyclohexylphosphino)butane, chlorodi(o-tolyl)phosphine, methyl diphenylphosphinite, di-tert-butylneopentylphosphonium tetrafluoroborate, 2-(di-tert-butylphosphino)dimethylaminobenzene, bis(3,5-di-tert-butyl-4-methoxyphenyl)chlorophosphine, Me₃(OMe)tBuXPhos, ethyldiphenylphosphine oxide, bis(3,5-bis(trifluoromethyl)phenyl)(2′,6′-bis(isopropoxy)-3,6-dimethoxybiphenyl-2-yl)phosphine, (1R,2R)-N,N′-bis└2-(diphenylphosphino)benzyl┘cyclohexane-1,2-diamine, bis(4-methylphenyl)chlorophosphine, bis(3,5-dimethyl-4-methoxyphenyl)chlorophosphine, tricyclopentylphosphine, 5-(Di-tert-butylphosphino)-1-(naphthalen-1-yl)-1H-pyrazole, bis[2-(diadamantylphosphino)ethyl]amine, isopropyldiphenylphosphine, divinylphenylphosphine, 2-(di-tert-butylphosphino)-1-(2-methoxyphenyl)-1H-pyrrole, 2-(1,1-dimethylpropyl)-6-(diphenylphosphino)pyridine, tricyclopentylphosphine tetrafluoroborate, tetraphenylbiphosphine, 5-(dicyclohexylphosphino)-1′,3′,5′-triphenyl-1′H-[1,4′]bipyrazole, 2′-dicyclohexylphosphino-2,4,6-trimethoxybiphenyl, triisopropylphosphonium tetrafluoroborate, (2-bromophenyl)dicyclohexylphosphine, (1R,2R)-2-(diphenylphosphino)cyclohexylamine, bis(2-methoxyphenyl)phosphine, tris(4-methyl-1-piperazinyl)phosphine, (S)-(4,4′,6,6′-tetramethoxybiphenyl-2,2′-diyl)bis(bis(3,5-di-tert-butyl-4-methoxyphenyl)phosphine, 2-(dicyclohexylphosphino)benzenesulfonic acid, 2-[bis(3,5-dimethylphenyl)phosphino]benzaldehyde, tert-butylphosphonic dichloride, 2-[di(2-methoxyphenyl)phosphino┐benzenesulfonic acid, (1R,2R)-2-Amino-1-phenylpropyldiphenylphosphine, bis(4-methoxyphenyl)chlorophosphine, isocyanatophosphonic dichloride, 2-(Diphenylphosphino)ethanaminium tetrafluoroborate, bis(3,5-di-tert-butyl-4-methoxyphenyl)phosphine, 2-[bis(3,5-di-tert-butyl-4-methoxyphenyl)phosphino]benzaldehyde, 9-[2-(diisopropylphosphino)phenyl]-9H-carbazole, (2-ammonioethyl)diisopropylphosphonium bis(tetrafluoroborate), (S)-1-(diphenylphosphino)-3-methyl-2-butylamine, ((4-trifluoromethyl)phenyl)di-tert-butylphosphine, bis[4-(1H,1H,2H,2H-perfluorodecyl)phenyl]phenylphosphine, tris(2,4-dimethyl-5-sulfanatophenyl)phosphine trisodium salt, 2-(dicyclohexylphosphino)-N,N-diisopropyl-1H-indole-1-carboxamide, bis(2-isopropoxyphenyl)chlorophosphine, bis(3,5-dimethyl-4-methoxyphenyl)chlorophosphine, di(1-adamantyl)-(2-triisopropylsiloxyphenyl)phosphine, 1,1′-bis[bis(dimethylamino)phosphino]ferrocene, tert-butylchloro(methyl)phosphine, diethyl 4-(trifluoromethyl)benzylphosphonate, 2-(diphenylphosphino)-N,N,N-trimethylbenzylammonium triflate, 1-methyl-2-(2-diphenylphosphinophenyl)-1H-benzoimidazole, triphenylphosphineimine hemisulfate salt, 2,2′-bis(diphenylphosphino)-1,1′-biphenyl, tetraisopropyl vinylidenediphosphonate, 2-(di-tert-butylphosphino)ethylamine, (R)-1-(diphenylphosphino)-3-methyl-2-butylamine, 3-(diphenylphosphino)propan-1-aminium tetrafluoroborate, (3-ammoniopropyl)di-tert-butylphosphonium bis(tetrafluoroborate), (2-ammonioethyl)di-tert-butylphosphonium bis(tetrafluoroborate), P,P-dichloroferrocenylphosphine, N-[2-(diphenylphosphino)benzylidene]cyclohexylamine, 2-(diphenylphosphino)benzaldehyde oxime, 2-(di-p-tolylphosphino)benzaldehyde, bis(2-furyl)phosphine chloride, 2′-(di-tert-butylphosphino)acetophenone ethylene ketal, 3-(di-tert-butylphosphonium)propane sulfonate, DPBP-bidentate phosphine, 4-(triphenylphosphonio)butane-1-sulfonate, bis(3,5-di(trifluoromethyl)phenyl)chlorophosphine, [1,3-phenylenebis(methylene)]bis(dicyclopentylphosphine), (9-benzyl-9-fluorenyl)dicyclohexylphosphonium tetrafluoroborate, tert-butyldimethylphosphine borane, tert-butyldicyclohexylphosphine, di-tert-butylmethylphosphine, phenylbis[4-(3,3,4,4,5,5,6,6,7,7,8,8,8-tridecafluorooctyl)phenyl]phosphine, tris[4-(tridecafluorohexyl)phenyl]phosphine, tert-Butyldiisopropylphosphine, 1,1′-bis(phenylphosphino)ferrocene, O-ethyl methylphosphonothioate, benzyldiphenylphosphine, dichloromethylphosphine, and 1,1′-bis(phenylphosphinidene)ferrocene.

In one specific aspect, the phosphine ligand is bis[(2-diphenylphosphino)phenyl]ether (DPEphos) or (9,9-Dimethyl-9H-xanthene-4,5-diyl)bis(diphenylphosphane) (also known as Xantphos).

### C. Examples

The following examples further illustrate this disclosure. The scope of the disclosure and claims is not limited by the scope of the following examples.

### 1. Precatalyst

A series of palladium sources were evaluated under a set of standard conditions to see which ones showed activity for carbonylative coupling of 1-heptene to octanoic acid to form normal or iso-octanoic anhydride (Table 1). The reactions were conducted using an equimolar amount of Xantphos to palladium and run in acetonitrile solvent under 40 atmospheres of CO pressure at 120° C. for 16 hours. Under these conditions, several palladium sources were found to be active pre-catalysts for the carboxycarbonylation reaction; highest yields were obtained using palladium(π-cinnamyl) chloride dimer, [Pd(cinnamyl)Cl]₂. This precatalyst was used for subsequent evaluation of ligands and reaction conditions.

TABLE 1

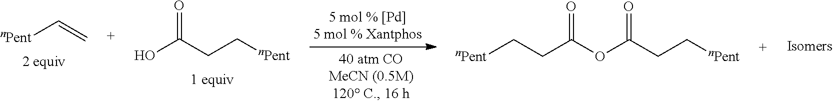

| | | | | | n- | i- | | |
|---|---|---|---|---|---|---|---|---|
| | Palladium | Conversion | Recovered | Recovered | Anhydride | Anhydride | Normal/Iso | |
| Entry | Precatalyst | (%) | n-Acid (%) | i-Acid (%) | Yield (%) | Yield (%) | Ratio | TON |
| 1 | Pd(OAc)₂ | — | — | — | 6 ^{a,b} | — | — | 1 |
| 2 | Pd(dba)₂ | — | — | — | 3 ^{a} | — | — | 1 |
| 3 | Pd(allyl)(IMes)Cl | — | — | — | trace | — | — | 0 |
| 4 | Pd(COD)(CH₂TMS)₂ | — | — | — | trace ^{c} | — | — | 0 |
| 5 | [Pd(allyl)TFA]₂ | 70 | 30 | — | 1^{c} | — | — | 0 |
| 6 | Pd(MeCN)₂(OTs)₂ | 71 | 29 | — | 1^{c} | — | — | 0 |
| 7 | Pd(MeCN)₄(BF4)₂ | — | — | — | 0^{c} | — | — | 0 |
| 8 | Xantphos Pd G3 ^{d} | 9 | 91 | — | 5^{c} | — | — | 1 |
| 9 | Xantphos Pd G4 ^{d} | 10 | 90 | — | 3^{c} | — | — | 1 |
| 10 | CpPd(cinnamyl) | 20 | 271 | — | 4.0 ^{c,e} | — | — | 0 |
| 11 | PdCl₂ | 73 | 236 | 17 | 44 ^{c,e} | 2 | 22 | 9 |
| 12 | Pd(PhCN)₂Cl₂ | 91 | 9 | — | 54.9 | 8.1 | 6.8 | 13 |
| 13 | Pd(MeCN)₂Cl₂ | 88 | 12 | — | 49.0 | 6.6 | 7.4 | 11 |
| 14 | Pd(PPh₃)₂Cl₂ | 96 | 4 | — | 66.4 | 14.0 | 4.8 | 16 |
| 15 | Pd(COD)Cl₂ | 91 | 9 | — | 57.7 | 10.3 | 5.6 | 14 |
| 16 | [Pd(allyl)Cl]₂ | 98 | 2 | — | 71.7 ^{b,f} | 15.5 | 4.6 | 9 |
| 17 | [Pd(cinnamyl)Cl]₂ | 97 | 3 | — | 73.3 ^{b,f} | 17.8 | 4.1 | 9 |
| 18 | [Pd(cinnamyl)Cl]₂ | 97 | 3 | — | 75 | 21 | 3.6 | 18 |
| 19 | Co₂(CO)₈ | 11 | 177 | — | 0 ^{c,e,g} | 0 | — | 0 |
| 20 | Ru₃(CO)₁₂ | 15 | 181 | — | 0 ^{c,e,g} | 0 | — | 0 |
| 21 | Mn₂(CO)₁₀ | 9 | 194 | — | 0 ^{c,e,g} | 0 | — | 0 |
| 22 | Fe₂(CO)₉ | 11 | 192 | — | 0 ^{c,e,g} | 0 | — | 0 |
| 23 | [Rh(cod)Cl]₂ | 12 | 200 | — | 0 ^{c,e,g} | 0 | — | 0 |
| 24 | [Rh(OAc)₂]₂ | 12 | 204 | — | 0 ^{c,e,g} | 0 | — | 0 |

^{a} Toluene used as solvent.
^{b} 10 mol % Xantphos used as ligand.
^{c} Reaction time of 3 hours.
^{d} Xantphos-ligated Buchwald carbazole palladium precatalyst generations 3 and 4.
^{e} Olefin to carboxylic acid stoichiometry of 1:3.
^{f} 5 mol % of palladium dimer used as precatalyst.
^{g} Chlorobenzene used as solvent with reaction run at 105° C.

### 2. Partial Pressure of Carbon Monoxide, Stoichiometry, and Temperature

Screening of different CO pressures showed 40 atm (589 psi) carbon monoxide as an efficient pressure (Table 2, entry 3). Lower pressures gave diminished yields (entries 1 and 2); however, higher pressures should have increased the yield (entry 4). The normal to iso ratio was essentially constant at all CO pressures screened. Evaluation of reagent stoichiometry suggested higher equivalents of the carboxylic acid was more efficient in producing higher yields of the normal anhydride isomer (Table 3, entry 8). Further analysis of the carboxylic acid mixtures recovered from the reaction indicates displacement of the iso anhydride isomer to produce symmetric anhydrides. Correcting for this displacement, when excess carboxylic acid is used, the regioselectivity of the hydropalladation step is not changed. However, with excess olefin, high yields of anhydride were observed, but no significant difference in normal-to-iso ratios was found. Furthermore, slightly lowering the temperature from 120° C. to 105° C. increased the normal to iso selectivity while not significantly affecting the overall yield (Table 4, entry 5). Further decreasing the temperature resulted in lower yields (Table 4, entries 1-4), and higher temperatures such as 135° C. decreased the selectivity towards anhydrides in favor of alkene isomerization (Table 4, entry 8).

TABLE 2

| | | | Recovered | Recovered | n-Anhydride | i-Anhydride | | |
|---|---|---|---|---|---|---|---|---|
| | CO | Conversion | n-Acid | i-Acid | Yield | Yield | Normal/Iso | |
| Entry | Pressure | (%) | (%) | (%) | (%) | (%) | Ratio | TON |
| 1 | 20 | 66 | 34 | 5 | 55 | 11 | 5 | 13 |
| 2 | 30 | 75 | 24 | 4 | 66 | 17 | 4 | 17 |
| 3 | 40 | 95 | 5 | 0 | 70 | 15 | 5 | 17 |
| 4 | 50 | 86 | 14 | 3 | 79 | 26 | 0 | 21 |

^{a} Reaction time of 3 hours.

TABLE 3

| | | | | Recovered | Recovered | n-Anhydride | i-Anhydride | Observed | Corrected | |
|---|---|---|---|---|---|---|---|---|---|---|
| | Olefin | Acid | Conversion ^{b} | n-Acid | i-Acid | Yield | Yield | Normal/Iso | Normal/Iso | |
| Entry | Equiv | Equiv | (%) | (%) | (%) | (%) | (%) | Ratio | Ratio | TON |
| 1 ^{c} | 14 | 1 | 100 | 0 | 0 | 77 | 23 | 3 | 3 | 20 |
| 2 | 5 | 1 | 100 | 0 | 0 | 77 | 23 | 4 | 4 | 19 |
| 3 | 3 | 1 | 100 | 0 | 0 | 72 | 20 | 4 | 4 | 18 |
| 4 | 2 | 1 | 95 | 5 | 0 | 70 | 15 | 5 | 5 | 17 |
| 5 | 1.5 | 1 | 79 | 21 | 3 | 68 | 24 | 3 | 2 | 18 |
| 6 | 1 | 1 | 71 | 29 | 4 | 58 | 20 | 3 | 2 | 16 |
| 7 | 1 | 1.5 | 91 | 62 | 6 | 72 | 20 | 4 | 3 | 18 |
| 8 | 1 | 2 | 91 | 107 | 9 | 78 | 17 | 5 | 3 | 19 |
| 9 | 1 | 3 | 93 | 200 | 13 | 86 | 11 | 8 | 3 | 19 |
| 10 | 1 | 5 | — | 411 | 15 | 61 | 5 | 12 | 2 | 13 |
| 11 ^{c} | 1 | 13 | — | 1139 | 31 | 86 | 3.5 | 25 | 2 | 18 |

^{a} Reaction time of 3 hours.
^{b} Conversion based on recovered limiting reagent.
^{c} Reaction run without solvent.

TABLE 4

| | Temperature | Conversion ^{b} | Recovered | Recovered | n-Anhydride | i-Anhydride | Normal/Iso | |
|---|---|---|---|---|---|---|---|---|
| Entry | (° C.) | (%) | n-Acid (%) | i-Acid (%) | Yield (%) | Yield (%) | Ratio | TON |
| 1 | 70 | 54 | 246 | 7 | 42 | 5 | 8 | 9 |
| 2 | 80 | 82 | 519 | 11 | 69 | 7 | 10 | 15 |
| 3 | 90 | 93 | 221 | 12 | 80 | 10 | 8 | 18 |
| 4 | 100 | 90 | 218 | 13 | 81 | 9 | 9 | 18 |
| 5 | 105 | 91 | 199 | 13 | 80 | 8 | 10 | 18 |
| 6 | 110 | 89 | 220 | 13 | 69 | 11 | 6 | 16 |
| 7 | 120 | 93 | 200 | 13 | 86 | 11 | 8 | 19 |
| 8^{c} | 135 | 47 | 43 | 11 | 35 | 16 | 2 | 10 |

^{a} Reaction time of 3 hours, with 3 equiv. of octanoic acid and 1 equiv. of heptene.
^{b} Conversion calculated based on recovered 1-heptene.
^{c}Used 1 equiv. octanoic acid and 2 equiv. of heptene.

### 3. Solvents

Most solvents screened had better solubility of the initial starting materials compared to acetonitrile (Table 5, entry 1). Halogenated and ethereal solvents improved solubility of the precatalyst and ligand (entries 2-8). Other aromatic solvents lead to diminished yields of anhydride (entries 9 and 10). Most solvents had linear to branched ratios similar to acetonitrile; however, perfluorotoluene (entry 6), tetrachloroethane (entry 7), and DMF (entry 12) all showed significant increase in selectivity for the normal isomer despite diminished yields. No reaction was observed with DMSO as solvent, although significant amount of olefin isomerization was detected (entry 13). When the reaction was run in the absence of solvent, anhydride yield was >70%, with a normal/iso ratio of 6 (entry 14). When octanoic acid was used in solvent quantities, the normal/iso ratio of anhydride was significantly improved due to the equilibrium of anhydride displacement with carboxylic acid (entry 15).

TABLE 5

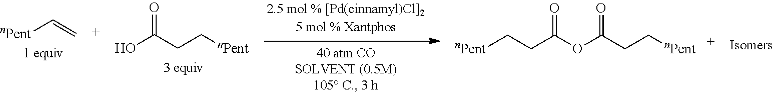

| | | Qualitative | Qualitative | | Recovered | Recovered | n- | i- | | |
|---|---|---|---|---|---|---|---|---|---|---|
| | | Initial | Final | Conversion ^{b} | n-Acid | i-Acid | Anhydride | Anhydride | Normal/Iso | |
| Entry | Solvent | Solubility | Solubility ^{a} | (%) | (%) | (%) | Yield (%) | Yield (%) | Ratio | TON |
| 1 | MeCN | Partly | Pd black, | 90 | 200 | 13 | 80 | 8 | 10 | 17 |
| | | soluble | other | | | | | | | |
| | | | precipitates | | | | | | | |
| 2 | PhCl | Fully | Less Pd | 97 | 210 | 12 | 81 | 11 | 7 | 18 |
| | | soluble | black | | | | | | | |
| 3 | DCM | Fully | Pd black | 99 | 203 | 11 | 80 | 10 | 8 | 18 |
| | | soluble | | | | | | | | |
| 4 | DCE | Fully | Pd black | 99 | 192 | 10 | 77 | 12 | 7 | 18 |
| | | soluble | | | | | | | | |
| 5 | PhCF₃ | Mostly | Pd black | 98 | 209 | 12 | 74 | 11 | 7 | 17 |
| | | soluble | | | | | | | | |
| 6 | toluene-F₈ | Partly | Pd black | 92 | 215 | 11 | 66 | 3 | 26 | 14 |
| | | soluble | | | | | | | | |
| 7 | tetrachloro- | Fully | Pd black | 36 | 269 | 3 | 12 | 0 | 60 | 2 |
| | ethane | soluble | | | | | | | | |
| 8 | THF | Fully | Less Pd | 65 | 246 | 11 | 47 | 5 | 9 | 10 |
| | | soluble | black | | | | | | | |
| 9 | PhCN | Fully | Pd black | 99 | 222 | 13 | 65 | 11 | 6 | 15 |
| | | soluble | | | | | | | | |
| 10 | toluene | Mostly | Pd black, | 72 | 231 | 10 | 50 | 7 | 7 | 11 |
| | | soluble | other | | | | | | | |
| | | | precipitates | | | | | | | |
| 11 | EtOAc | Mostly | Less Pd | 95 | 212 | 11 | 58 | 8 | 7 | 13 |
| | | soluble | black | | | | | | | |
| 12 | DMF | Fully | Less Pd | 73 | 231 | 10 | 41 | 2 | 27 | 8 |
| | | soluble | black | | | | | | | |
| 13 | DMSO | Fully | Pd black | 67 | 291 | 5 | 0 | 0 | — | 0 |
| | | soluble | | | | | | | | |
| 14 | none ^{c} | Mostly | Pd black, | 98 | 191 | 11 | 64 | 10 | 6 | 15 |
| | | soluble | other | | | | | | | |
| | | | precipitates | | | | | | | |
| 15 | Octanoic | Partly | Yellow | — | 1139 | 31 | 86 | 4 | 25 | 18 |
| | acid | soluble | precipitates | | | | | | | |

^{a} Precipitates soluble in DCM during workup.
^{b} Conversion calculated based on recovered 1-heptene.
^{c} Reaction run in absence of solvent.

### 4. Phosphine Ligands

A variety of different ligand types were screened for catalytic activity for the carbonylation of 1-heptene in acetonitrile solvent (Table 6). Under these conditions, in acetonitrile solvent, ligands afforded anhydrides ranging from trace amounts to over 70%.

TABLE 6

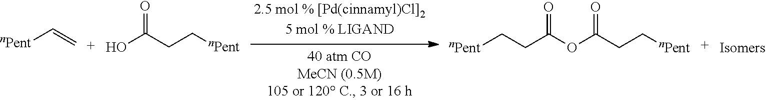

Conditions: (A) 1 equiv acid, 2 equiv olefin, 120° C. (B) 1 equiv olefin, 3 equiv acid, 105° C.

| | | | Con- | Recovered | Recovered | n- | i- | | | |
|---|---|---|---|---|---|---|---|---|---|---|
| | | Condition/ | version ^{a} | n-Acid | i-Acid | Anhy- | Anhy- | n/i | Corrected | |
| Entry | Ligand | Time (h) | (%) | (%) | (%) | dride | dride | Ratio | n/i Ratio | TON |
| 1 | 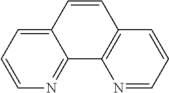 [SMILES: c1cnc2c(c1)ccc1cccnc12] | A/16 | — | — | — | trace | — | — | — | — |
| 2 | 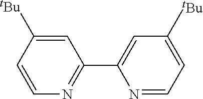 [SMILES: CC(C)(C)c1ccnc(-c2cc(C(C)(C)C)ccn2)c1] | A/16 | — | — | — | trace | — | — | — | — |
| 3 | 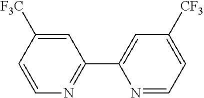 [SMILES: FC(F)(F)c1ccnc(-c2cc(C(F)(F)F)ccn2)c1] | A/16 | — | — | — | trace | — | — | — | — |
| 4 | 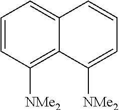 [SMILES: CN(C)c1cccc2cccc(N(C)C)c12] | A/16 | — | — | — | trace | — | — | — | — |
| 5 | PPh₃ | A/16 | 85 | 15 | 0 | 24.1 | 7.5 | 3.2 | 3.2 | 6 |
| 6 | P(C₆F₅)₃ | A/16 | — | — | — | trace | — | — | — | — |
| 7 | 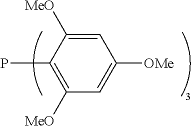 | A/16 | — | — | — | trace | — | — | — | — |
| 8 | 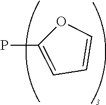 | A/16 | 83 | 17 | 0 | 20.5 | 5.9 | 3.5 | 3.5 | 5 |
| 9 | PBn₃ | A/16 | 82 | 18 | 0 | 16.8 | 3.8 | 4.4 | 4.4 | 4 |
| 10 | P(^{t}Bu)₃ | A/16 | — | — | 0 | trace | — | — | — | — |
| 11 | PBu(Ad)₂ | A/16 | 83 | 17 | 0 | 26.7 | 5.8 | 4.6 | 4.6 | 7 |
| 12 | 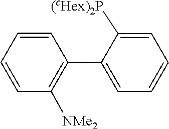 | A/16 | — | — | — | trace | — | — | — | — |
| 13 | 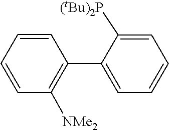 | A/16 | — | — | — | trace | — | — | — | — |
| 14 | 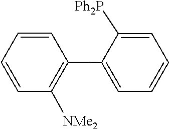 | A/16 | 82 | 18 | 0 | 5.9 | 0.3 | 19.7 | 19.7 | 1 |
| 15 | 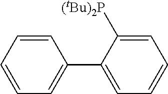 | A/16 | — | — | — | trace | — | — | — | — |
| 16 | 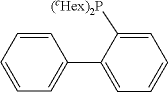 | A/16 | 79 | 21 | 0 | 17.5 | 2.9 | 6.0 | 6.0 | 4 |
| 17 | 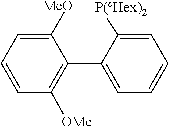 | A/16 | 75 | 25 | 0 | 8.1 | 0.7 | 11.6 | 11.6 | 2 |
| 18 | 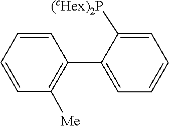 | A/16 | — | — | — | trace | — | — | — | — |
| 19 | 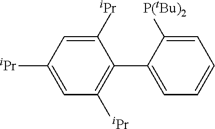 | A/16 | 81 | 19 | 0 | 16.6 | 3.2 | 5.2 | 5.2 | 4 |
| 20 | 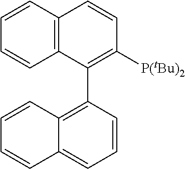 | A/16 | — | — | — | trace | — | — | — | — |
| 21 | 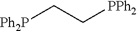 | A/16 | — | — | — | trace | — | — | — | — |
| 22^{b} | 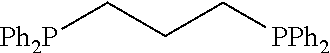 | A/16 | 81 | 19 | 0 | 24.2 | 0.4 | 60.5 | 60.5 | 5 |
| 23 | 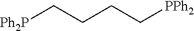 | A/16 | 79 | 21 | 0 | 19.5 | 0.9 | 21.7 | 21.7 | 4 |
| 24 | rac-BINAP | A/16 | 81 | 19 | 0 | 23.5 | 0.3 | 78.3 | 78.3 | 5 |
| 25 | 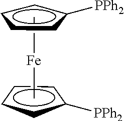 | A/16 | 91 | 9 | 0 | 53.7 | 5.7 | 9.4 | 9.4 | 12 |
| 26 | 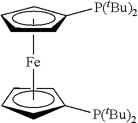 | A/16 | — | — | — | trace | — | — | — | — |
| 27 | 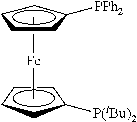 | A/16 | 91 | 9 | 0 | 50.1 | 3.3 | 15.2 | 15.2 | 11 |
| 28 | 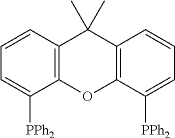 | A/16 | 97 | 3 | 0 | 74 | 21 | 3.5 | 3.5 | 19 |
| 29 30 | 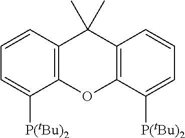 | A/3 A/16 | — — | — — | — — | trace trace | — — | — — | — — | — — |
| 31 32 | 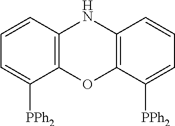 | A/3 A/16 | 90 96 | 10 4 | 0 0 | 56 64 | 10 16 | 6 4 | 6 4 | 13 16 |
| 33 34 35 | 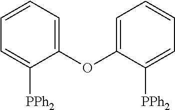 | A/16 A/3 B/16 | 91 87 | 9 25 218 | 0 0 16 | 43 15 65 | 12 1 7 | 4 15 9 | 15 2 | 11 3 14 |
| 38 39 | 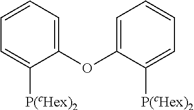 | B/3 B/16 | 12 31 | 287 268 | 0 2 | 1.2 6 | 0 0.3 | — 20 | — 2 | 10 |
| 36 37 | 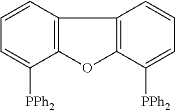 | A/3 A/16 | | 31 21 | 0 0 | 2 18 | 0 1 | — 18 | — 18 | 0 4 |
| 40 41^{c} | 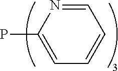 | B/3 B/16 | 20 52 | 249 266 | 0 4 | 0.4 25 | 0 1.4 | — 18 | — 4 | 0 5 |
| 42 43 | 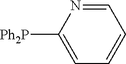 | B/3 B/16 | 25 67 | 260 249 | 5 16 | 15 32 | 1.1 5.5 | 14 6 | 2 1 | 3 8 |
| 44 45 | 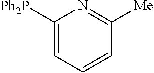 | B/3 B/16 | 21 51 | 285 254 | 4 12 | 9 28 | 0 2.6 | — 11 | 1 1 | 2 6 |
| 46 47^{c} | 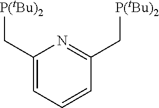 | B/3 B/16 | 8 26 | 298 282 | 0 0 | 0.1 0.5 | 0 0 | — — | — — | 0 0 |
| 48 49 | 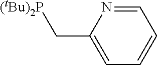 | B/3 B/16 | 6 30 | 294 275 | 0 0 | 0.4 0.2 | 0 0 | — — | — — | 0 0 |
| 50 | 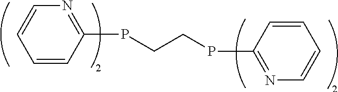 | B/3 | 31 | 248 | 0 | 0.8 | 0 | — | — | 0 |
| 51 52 | 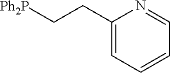 | B/3 B/16 | 2 19 | 291 289 | 0 0 | trace 0.5 | 0 | — — | — — | 0 |
| 53 54 | 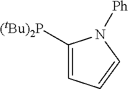 | B/3 B/16 | 7 30 | 293 292 | 0 1 | 0.2 4.9 | 0 0 | — — | — 4 | 0 1 |
| 55 | 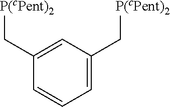 | B/3 | 9 | 294 | 0.6 | 1.4 | 0 | — | 1.3 | 0 |
| 56 57 | 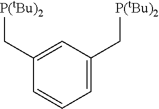 | B/3 B/16 | 17 30 | 289 271 | 0.8 5 | 1.3 11 | 0 0.8 | — 14 | 0.6 1 | 0 2 |
| 58 59 | 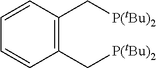 | B/3 B/16 | 48 65 | 261 240 | 0 3 | 39 36 | 0.6 1.5 | 65 24 | 65 7 | 8 8 |
| 60 | 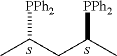 | B/3 | 12 | 286 | 0 | 0.4 | 0 | — | — | 0 |
| 61 | 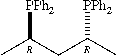 | B/3 | 7 | 286 | 0 | 0.5 | 0 | — | — | 0 |
| 62 | 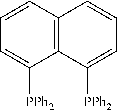 | B/3 | 10 | 288 | 0 | 0.2 | 0 | — | — | 0 |
| 63 | BIPHEPHOS | B/3 | 25 | 295 | 3 | 8 | 0 | — | 2 | 2 |
| 64 | (−)-DIPAMP | B/3 | 14 | 284 | 0 | 0.1 | 0 | — | | 0 |
| 65^{d} | (R,R)-DIOP | B/3 | 96 | 208 | 5 | 47 | 1.6 | 29 | 6.4 | 10 |
| 66 | | B/16 | 99 | 241 | 12 | 48 | 2.4 | 20 | 2.5 | 10 |
| 67 | F1 | B/3 | 10 | 294 | 0 | 2.8 | 0 | — | — | 1 |
| 68 | | B/16 | 37 | 282 | 2 | 13 | 0.2 | 65 | 5 | 3 |
| 69 | F2 | B/3 | 10 | 297 | 0 | 0.5 | 0 | — | — | 0 |
| 70 | | B/16 | 33 | 280 | 0.4 | 2 | 0 | — | 4 | 0 |
| 71 | F3 | B/3 | 11 | 291 | 0 | 2.9 | 0 | — | — | 1 |
| 72 | | B/16 | 58 | 254 | 9 | 32 | 1.6 | 20 | 2 | 7 |
| 73 | F4 | B/3 | 14 | 287 | 0 | 0.8 | 0 | — | — | 0 |
| 74 | | B/16 | 35 | 272 | 3 | 16 | 0.3 | 53 | 4 | 3 |
| 75 | F5 | B/3 | 17 | 285 | 0 | 0.2 | 0 | — | — | 0 |
| 76 | | B/16 | 35 | 282 | 0.2 | 0.8 | 0 | — | 3.0 | 0 |

^{a} Conversion based on recovered limiting reagent.
^{b} 5 mol % palladium allyl chloride dimer used as precatalyst.
^{c} 10 mol % of ligand used.
^{d} Chlorobenzene used as solvent.
Ad = adamantly, ^{c}Pent = cyclopentyl.

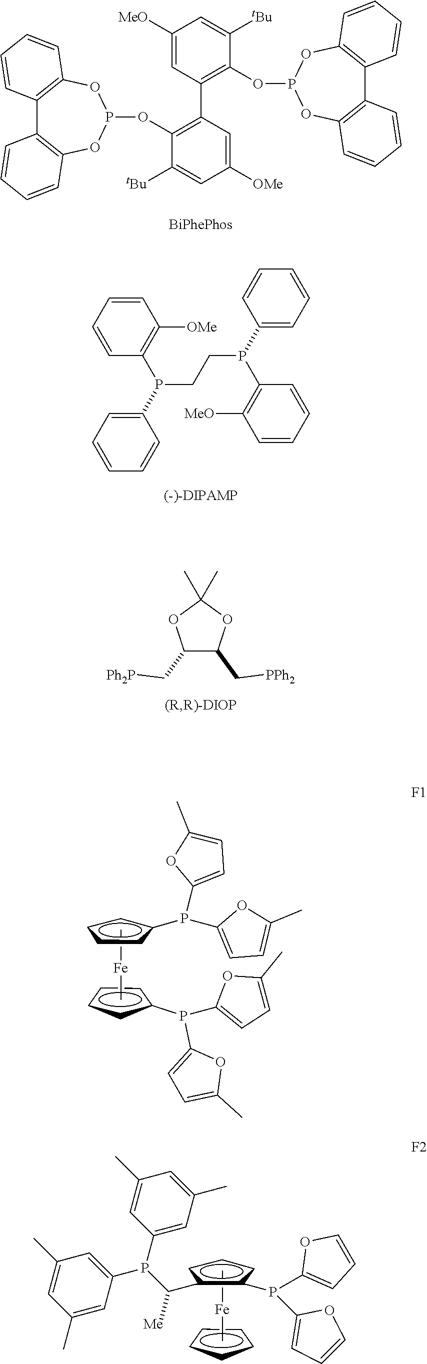

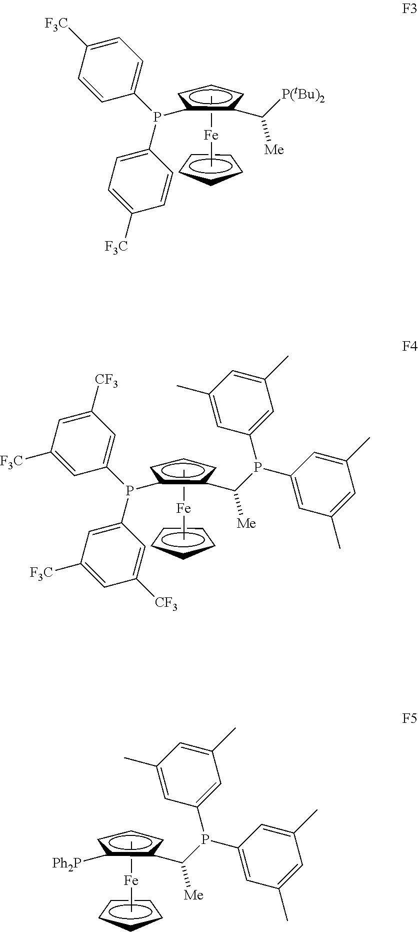

TABLE 7

| | | Recovered | Recovered | | | | |
|---|---|---|---|---|---|---|---|
| | | n-Acid | i-Acid | | | Observed | Corrected |
| Entry | Ligand | (%) | (%) | n-Anhydride | i-Anhydride | Normal/Iso | Normal/Iso |
| 1 | Xantphos | 186 | 9 | 72 | 6 | 12 | 4 |
| 2 | 2-PPh₂-pyridine | 288 | 3 | 7 | 0 | — | 1 |
| 3 | 2-P(tBu)₂-NPh-pyrrolc | 294 | 3 | 12 | 0 | — | 3 |
| 4 | P(2-furyl)₃ | 260 | 14 | 24 | 3 | 8 | 1 |
| 5 | P(p-An)₃ | 280 | 5 | 9 | 0 | — | 1 |
| 6 | P(p-Tol)₃ | 292 | 7 | 15 | 0 | — | 1 |
| 7 | P(Bn)₃ | 300 | 2 | 5 | 0 | — | 2 |

^{a}Same conditions as Table 6.

### 5. Co-Catalytic Additives

Addition of chloride additives such as cinnamyl chloride and tetrabutylammonium chloride (TBACl) have offered minor improvements in reaction yield (Table 8, entries 2 and 3). When a catalytic amount of p-toluenesulfonic acid (PTSA) was added, a decrease in yield was observed (entry 4). Palladium black and other precipitates are typically observed in the above reactions, and this problem was minimized when benzoyl chloride (BzCl) was added in catalytic amounts (entry 5). With 5 mol % BzCl, the loading of palladium precatalyst can be further lowered to 1 or 0.5 mol % (entries 6 and 7). Further changing palladium:ligand ratios to include excess ligand also helped to minimize the amount of palladium black formed (entries 8, 9, and 10). Lower yields were observed without BzCl additive at lower catalyst loadings (entry 11), and the reaction shut down in the absence of BzCl with excess ligand (entry 12). Changing equivalents of BzCl additive do not improve yield or greatly affect normal/iso selectivity (entries 13 and 14).

Reviewing chloride additives at lower palladium loadings with excess xantphos revealed acyl and cinnamyl chlorides (entries 15 and 16) were superior to simple chloride salts like TBACl and lithium chloride (entries 17 and 18), although lithium chloride as an additive nearly doubled the normal/iso ratio. A combination of BzCl and lithium chloride was attempted to improve yield and selectivity; however, there was no improvement over just BzCl as an additive (entry 19). Other acyl additives acetic anhydride and trifluoroacetic anhydride (TFAA) produced anhydride in diminished yields, but higher normal/iso selectivity was seen with acetic anhydride additive (entries 20 and 21). Since BzCl and carboxylic acids are known to readily produce anhydrous HCl, other sources of acid were added in catalytic amounts. However, anhydrous HCl in dioxane and other sulfonic acids all produced lower yields of anhydride (entries 22-25). Palladium(II) chloride is an active precatalyst under these conditions, and its activity is greatly enhanced when used in combination with a catalytic amount of benzoyl chloride (entries 26-27).

TABLE 8

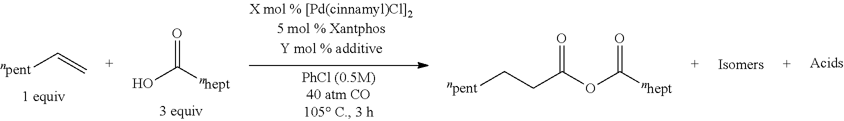

| | Precatalyst | Pd:Ln | | Amount | n-Anhydride | i-Anhydride | Normal/Iso | |
|---|---|---|---|---|---|---|---|---|
| Entry | loading | Ratio | Additive | (mol %) | Yield (%) | Yield (%) | Ratio | TON |
| 1 | 2.5 | 1:1 | none | — | 72 | 6 | 12 | 16 |
| 2 | 2.5 | 1:1 | cinnamyl chloride | 5 | 77 | 8 | 10 | 17 |
| 3 | 2.5 | 1:1 | TBACl | 5 | 78 | 9 | 9 | 17 |
| 4 | 2.5 | 1:1 | PTSA | 5 | 44 | 6 | 7 | 10 |
| 5 | 2.5 | 1:1 | BzCl | 5 | 74 | 10 | 7 | 17 |
| 6 | 1 | 1:1 | BzCl | 5 | 80 | 6 | 13 | 43 |
| 7 | 0.5 | 1:1 | BzCl | 5 | 72 | 6 | 12 | 78 |
| 8 | 1 | 1:2 | BzCl | 5 | 77 | 9 | 9 | 43 |
| 9 | 1 | 2:5 | BzCl | 5 | 79 | 8 | 10 | 43 |
| 10 | 0.5 | 1:5 | BzCl | 5 | 73 | 4.3 | 17 | 77 |
| 11 | 1 | 1:1 | none | — | 50 | 4 | 13 | 27 |
| 12 | 1 | 2:5 | nonc | — | 4 | 0 | — | 2 |
| 13 | 1 | 2:5 | BzCl | 2.5 | 74 | 7 | 11 | 41 |
| 14 | 1 | 2:5 | BzCl | 10 | 81 | 10 | 8 | 6 |
| 15 | 1 | 2:5 | AcCl | 5 | 76 | 11 | 7 | 43 |
| 16 | 1 | 2:5 | cinnamyl chloride | 5 | 74 | 6.0 | 12 | 40.0 |
| 17 | 1 | 2:5 | TBACl | 5 | 8 | 0 | — | 4.0 |
| 18 | 1 | 2:5 | LiCl | 5 | 40 | 2 | 20 | 21 |
| 19 | 1 | 2:5 | BzCl + LiCl | 5 + 5 | 78 | 8 | 10 | 43 |
| 20 | 1 | 2:5 | Ac₂O | 5 | 34 | 0.7 | 49 | 17 |
| 21 | 1 | 2:5 | TFAA | 5 | 61 | 5 | 12 | 33 |
| 22 | 1 | 2:5 | 4 M HCl in dioxane | 5 | 23 | 2 | 12 | 13 |
| 23 | 1 | 2:5 | PTSA | 5 | 46 | 6 | 8 | 26 |
| 24 | 1 | 2:5 | PhSO₃H | 5 | 34 | 0 | — | 17 |
| 25 | 1 | 2:5 | MeSO₃H | 5 | 24 | 0 | — | 12 |
| 26 | 2 | 2:5 | none | — | 8 | 0 | — | 2 |
| 27 | 2 | 2:5 | BzCl | 5 | 43 | 2 | 22 | 11 |

TABLE 9

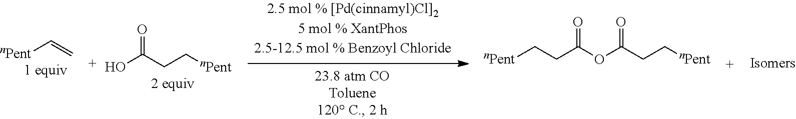

| Entry | Additive | Reaction Time | Normal Yield (%) | Iso Yield (%) |
|---|---|---|---|---|
| 1 | 0 mol % Benzoyl chloride | 2 | 22 | — |
| 2 | 2.5 mol % Benzoyl chloride | 2 | 28 | — |
| 3 | 5.0 mol % Benzoyl chloride | 2 | 34 | — |
| 4 | 7.5 mol % Benzoyl chloride | 2 | 30 | — |
| 5 | 10.0 mol % Benzoyl chloride | 2 | 30 | — |
| 6 | 12.5 mol % Benzoyl chloride | 2 | 32 | — |
| 7 | 5.0 mol % Benzoyl chloride | 6 | 25 | — |

Concentration of reagents: P_{CO} = 23.8 atm, 0.1 M Heptene, 0.2 M Octanoic Acid, 0.0025 M Pd(cinnamyl)Cl₂, 0.005 M Xantphos.

TABLE 10

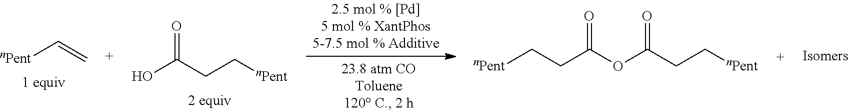

| Entry | Catalyst/Ligand | Additive | Normal Yield (%) | Iso Yield (%) |
|---|---|---|---|---|
| 1 | Pd(OAc)₂/Xantphos | 5.0 mol % Tetrabutylammonium Chloride | — | — |
| 2 | Pd(OAc)₂/Xantphos | 5.0 mol % Tetraethylammonium Bromide | — | — |
| 3 | Pd(OAc)₂/Xantphos | 5.0 mol % Tetrabutylammonium Iodide | — | — |
| 4 | Pd(OAc)₂/Xantphos | 7.5 mol % Benzoyl Bromide | 49 | — |
| 5 | Pd(cinnamyl)Cl]₂/Xantphos | 5.0 mol % Benzoyl Bromide | 21 | — |
| 6 | [Pd(cinnamyl)Cl]₂/Xantphos | 5.0 mol % Cesium Iodide | 29 | — |
| 7 | [Pd(cinnamyl)Cl]₂/Xantphos | 5.0 mol % Cesium Bromide | 16 | — |
| 8 | Pd(OAc)₂/Xantphos | 5.0 mol % Methyl Iodide | — | — |
| 9 | Pd(OAc)₂/Xantphos | 7.5 mol % Methyl Iodide | — | — |
| 10 | Pd(OAc)₂/Xantphos | 7.5 mol % Cesium Iodide | — | — |
| 11 | Pd(OAc)₂/Xantphos | 7.5 mol % Cesium Bromide | — | — |
| 12 | [Pd(cinnamyl)Cl]₂/Xantphos | 5.0 mol % Methyl Iodide | 21 | — |
| 13 | [Pd(cinnamyl)Cl]₂/Xantphos | 7.5 mol % Methyl Iodide | 20 | — |
| 14 | Pdl₂/Xantphos | 7.5 mol % Benzoyl Bromide | 1^{a} | — |
| 15 | Pdl₂/Xantphos | 7.5 mol % 4-Iodobenzotrifluoride | — ^{a} | — |
| 16 | Pdl₂/Xantphos | 7.5 mol % Methyl Iodide | — ^{a} | — |
| 17 | Pdl₂/Xantphos | 7.5 mol % 1-Todooctane | — ^{a} | — |
| 18 | Pdl₂/Xantphos | 7.5 mol % Lithium Iodide | — ^{a} | — |

Concentration of reagents: P_{CO} = 23.8 atm, 0.1 M Heptene, 0.2 M Octanoic Acid, 0.0025 M Pd(OAc)₂/Pd(cinnamyl)Cl₂/PdI₂, 0.005 M Xantphos. ^{a} P_{CO} = 11.9 atm

TABLE 11

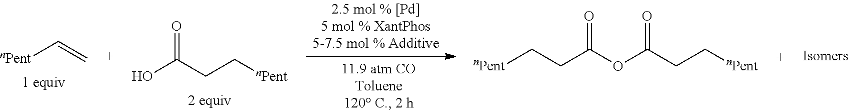

| | | | | Iso |
|---|---|---|---|---|
| | | | Normal Yield | Yield |
| Entry | Catalyst/Ligand | Additive | (%) | (%) |
| 1 | Pd(OAc)₂/Xantphos | 7.5 mol % Benzoyl Chloride | 5 | — |
| 2 | Pd(OAc)₂/Xantphos | 7.5 mol % Benzoyl Bromide | 68 | — |
| 3 | Pd(OAc)₂/Xantphos | 7.5 mol % Methyl Iodide | — | — |
| 4 | [Pd(cinnamyl)Cl]₂/Xantphos | 7.5 mol % Benzoyl Chloride | 3 | — |
| 5 | Pd(OAc)₂/Xantphos | 5.0 mol % Benzoyl Bromide | 20^{a} | — |
| 6 | Pd(OAc)₂/Xantphos | 7.5 mol % Benzoyl Bromide | 18^{a} | — |
| 7 | Pd(OAc)₂/Xantphos | 5.0 mol % Benzoyl Bromide | 31^{a} | — |
| 8 | Pd(OAc)₂/Xantphos | 7.5 mol % Benzoyl Bromide | 11^{a} | — |

Concentration of reagents: P_{CO} = 11.9 atm, 0.1 M Heptene, 0.2 M Octanoic Acid, 0.0025 M Pd(OAc)₂/[Pd(cinnamyl)Cl]₂, 0.005 M Xantphos. ^{a} P_{CO} = 5.95 atm

TABLE 12

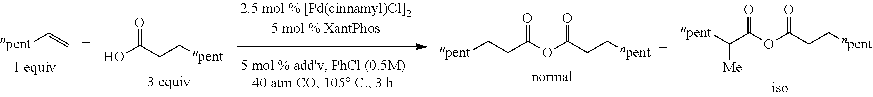

| | | n-Acid | i-Acid | n-Anhydride | i-Anhydride | n/i | Corrected | |
|---|---|---|---|---|---|---|---|---|
| Entry | Additive | (%) | (%) | (%) | (%) | Ratio | n/i | TON |
| 1 | None | 283 | 0 | 4 | 0 | — | — | — |
| 2 | BzCl | 202 | 11 | 81 | 8 | 10 | 4 | 44.5 |
| 3 | AcCl | 210 | 12 | 76 | 11 | 7 | 3 | 43.5 |
| 4 | cinnamyl chloride | 187 | 7 | 74 | 6 | 12 | 5 | 40.0 |
| 5 | TBACl | 301 | 0 | 8 | 0 | >20 | — | — |
| 6 | LiCl | 262 | 7 | 40 | 2 | 20 | 4 | 21.0 |
| 7 | BzCl + LiCl | 190 | 11 | 78 | 8 | 10 | 4 | 43.0 |
| 8 | PhI | 232 | 26 | 39 | 9 | 4 | 0.37 | 24.0 |
| 9 | PhI | 7 | 0 | 83 | 8 | 10 | 10 | 45.5 |
| 10 | Ac2O | 242 | 4 | 26 | 1 | 43 | 5 | 13.3 |
| 11 | TFAA | 226 | 11 | 61 | 5 | 12 | 3 | 33.0 |
| 12 | HCl | 263 | 18 | 23 | 2 | 12 | 0.25 | 12.5 |
| 13 | HCl | 256 | 29 | 38 | 6 | 6 | 0.26 | 22.0 |
| 14 | PTSA | 246 | 19 | 46 | 6 | 8 | 1.1 | 26.0 |
| 15 | PhSO3H | 357 | 10 | 34 | 0 | — | 2 | — |
| 16 | MeSO3H | 219 | 32 | 24 | 0 | — | — | — |
| 17 | BzOH | 261 | 6 | 46 | 3 | 15 | 4 | 24.5 |
| 18 | PivOH | 268 | 4 | 23 | 1 | 23 | 4 | 12.0 |
| 19 | 4 Å MS | 302 | 0 | 0 | 0 | — | — | — |

### 6. Propylene Reactions

Propylene was examined as the olefin for the production of symmetric C4 anhydrides with butyric acid and asymmetric C4:C8 anhydrides with octanoic acid. The overwhelming majority of the anhydride product is the symmetric product corresponding to the acid present; C8:C8 for the propylene and octanoic acid runs and C4:C4 for the propylene butyric acid runs (Table 13).

TABLE 13

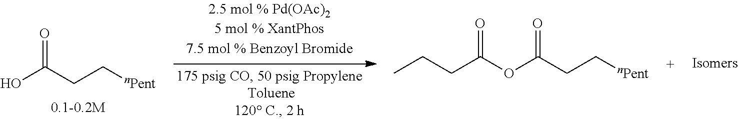

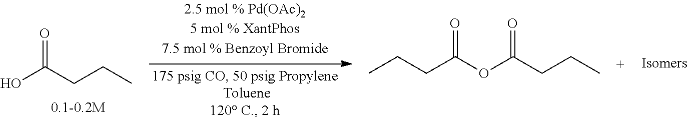

| | | C4 | C4:C8 | C8 | C4 Anhydride |
|---|---|---|---|---|---|
| Cat/Lig | Acid | Anhydride | Anhydride | Anhydride | n/i ratio |
| Pd(OAc)₂/xant | 0.1M Octanoic Acid | 0.015M | 0.0008M | 0.049M | 2.5 |
| Pd(OAc)₂/xant | 0.15M Octanoic | 0.030M | 0.0018M | 0.078M | 3.3 |
| | Acid | | | | |
| Pd(OAc)₂/xant | 0.2M Octanoic Acid | 0.020M | 0.0011M | 0.097M | 3.4 |
| Pd(OAc)₂/xant | 0.1M Butyric Acid | 0.061M | — | — | 9.4 |
| Pd(OAc)₂/xant | 0.15M Butyric Acid | 0.068M | — | — | 13.6 |
| Pd(OAc)₂/xant | 0.2M Butyric Acid | 0.113M | — | — | 17.9 |

Concentration of reagents: P_{CO} = 175 psig, P_{C3H6} = 50 psig, 0.0025M Pd(OAc)₂, 0.005M Xantphos.

### 7. Propylene Reactions at Lower Pressure

Propylene was examined at lower CO, and propylene pressure as well as a range of temperatures. At 120° C., 25 psig propylene, and 50 psig CO, anhydride yield was low due to the formation of a significant amount of heptene that was quickly isomerized to the internal olefins, trapping the majority of the octanoic acid in an inactive form. The temperature was lowered to 105 and 90° C. to suppress the isomerization of 1-heptene resulting in an improvement in anhydride yield at the lower temperatures. The major product was the symmetric C8 acid dehydration condensation species, with a small amount of the symmetric C4 anhydrides (linear and branched) and almost none of the asymmetric C4:C8 species.

TABLE 14

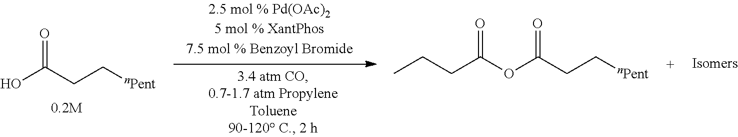

| | Temperature | Pressure | Anhydride | C4:C8 | Anhydride | 1-Heptene |
|---|---|---|---|---|---|---|
| Entry | (° C.) | (atm) | C4 | Anhydride | C8 | (isomers) |
| 1 | 120 | 1.7 Propylene | 0.008M | — | 0.011M | 0.024M |
| | | 3.4 CO | | | | (0.087M) |
| 2 | 105 | 1.7 Propylene | 0.012M | — | 0.086M | 0.029M |
| | | 3.4 CO | | | | (0.018M) |
| 3 | 90 | 0.7 Propylene | 0.002M | — | 0.089M | 0.004M |
| | | 3.4 CO | | | | (—) |
| 4^{a} | 90 | 0.7 Propylene | 0.001M | 0.006M | 0.061M | 0.003M |
| | | 3.4 CO | | | | (—) |
| 5^{b} | 90 | 0.7 Propylene | — | — | 0.003M | — |
| | | 3.4 CO | | | | (—) |

Concentration of reagents: P_{CO} = 3.4 atm, P_{C3H6} = 0.7-1.7 atm, 0.0025 M Pd(OAc)₂, 0.005 M Xantphos.
^{a}added 0.2 mol % Rh.
^{b}added 2.0 mol % Rh.

### 8. Propylene Reactions at Higher Pressures

The following standard conditions were examined first with n-butyric acid: 0.5 mol % ┌Pd(cinnamyl)Cl┐₂, 2 mol % PPh₃ and 5 mol % benzoyl chloride, under 40 atm of CO and propylene (p_{CO}=31.2 atm, p_{propylene}=8.8 atm), in DCM solvent. Under these conditions, with 1.33 M n-butyric acid, a mixture of anhydrides at a concentration of 0.48 M was obtained (Table 15; Entry 1). This reaction produced almost equal amounts of branched and linear products. Running the reaction for 22 hours instead of 3 hours, a significant amount more isobutyric acid and isobutyric anhydride was formed through post-catalytic scrambling of acid (Table 15; Entry 2). Changing the additive from benzoyl chloride to hydrogen chloride did not improve the reaction yield, but it did decrease branched selectivity (Table 15; Entry 3). We tested the reactivity of xantphos and Dtbpx (Table 15; Entries 4 and 6) in the presence of benzoyl chloride. Changing the additive to HCl·Et₂O slightly increased linear selectivity for xantphos but not for Dtbpx ligand (Table 15; Entries 5 and 7). Decreasing the pressure to 20 atm drastically reduced the yield (Table 15; Entry 8).

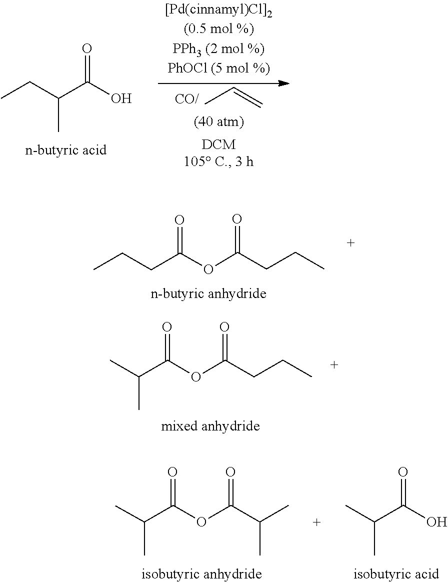

TABLE 15

| | Deviation | | | | n- | | | | |
|---|---|---|---|---|---|---|---|---|---|
| | from | n-butyric | Mixed | Isobutyric | butyric | | | | |
| | Standard | anhydride | anhydride | anhydride | acid | Isobutyric | Anhydrides | Total | Total |
| Entry | Conditions | (M) | (M) | (M) | (M) | acid (M) | (L/B) | L/B | TON |
| 1 | None | 0.25 | 0.19 | 0.04 | 0.47 | 0.18 | 2.6 | 2.6 | 35 |
| 2 | 22 h | 0.27 | 0.28 | 0.07 | 0.37 | 0.19 | 1.9 | 1.9 | 47 |
| | instead of 3 | | | | | | | | |
| | h | | | | | | | | |
| 3 | HCl•Et₂O | 0.21 | 0.06 | 0.00 | 0.89 | 0.11 | 6.9 | 7.7 | 21 |
| | instead of | | | | | | | | |
| | benzoyl | | | | | | | | |
| | chloride | | | | | | | | |
| 4 | xantphos | 0.50 | 0.33 | 0.06 | 0.45 | 0.15 | 2.9 | 2.9 | 67 |
| | instead of | | | | | | | | |
| | PPh₃ | | | | | | | | |
| 5 | xantphos | 0.41 | 0.10 | 0.01 | 0.63 | 0.07 | 8.0 | 8.5 | 39 |
| | and | | | | | | | | |
| | HCl•Et₂O | | | | | | | | |
| | instead of | | | | | | | | |
| | PPh₃ and | | | | | | | | |
| | benzoyl | | | | | | | | |
| | chloride | | | | | | | | |
| 6 | Dtbpx | 0.78 | 0.05 | 0.00 | 0.45 | 0.02 | 31.0 | 29.8 | 62 |
| | instead of | | | | | | | | |
| | PPh₃ | | | | | | | | |
| 7 | Use of | 0.61 | 0.06 | 0.00 | 0.56 | 0.02 | 21.4 | 24.2 | 50 |
| | Dtbpx and | | | | | | | | |
| | HCl•Et₂O | | | | | | | | |
| | instead of | | | | | | | | |
| | PPh₃ and | | | | | | | | |
| | benzoyl | | | | | | | | |
| | chloride | | | | | | | | |
| 8 | 20 atm | 0.08 | 0.02 | 0.00 | 1.16 | 0.13 | 8.7 | 9.1 | 8 |
| | instead of | | | | | | | | |
| | 40 atm | | | | | | | | |

Reaction conditions: [Pd(cinnamyl)Cl]₂ (0.5 mol %), PPh₃ (2 mol %), butyric acid (1.5 mmole, 1.33M), CO/propylene (40 atm) [p_{propylene} = 8.8 atm, p_{CO} = 31.2 atm], 105° C., 5 mol % benzoyl chloride, 3 h. Molarity was calculated by quantitative ¹³C NMR with HMDSO as an internal standard. TON = (n-butyric anhydride + mixed anhydride + isobutyric anhydride)/mmole of catalyst. Anhydride L/B = ((2*n-butyric anhydride) + mixed anhydride)/(mixed anhydride + (2*isobutyric anhydride)) and total L/B = ((2*n-butyric anhydride) + mixed anhydride + linear acid)/(mixed anhydride + (2*isobutyric anhydride) + isobutyric acid).

Reactivity with isobutyric was also explored, with standard reaction conditions yielding high branched selectivity with moderate TON (Table 16; Entry 1). Lower branched selectivity was observed at longer reaction times (Table 16; Entry 2). Using xantphos, both high activity and branched selectivity were observed (TON =104) (Table 16; Entry 3). However, when the reaction was performed with Dtbpx, the combined anhydride concentration was only 0.75 M (Table 16; Entry 4), whereas with xantphos the combined anhydride concentration was 1.37 M. The increased steric hindrance of the ligand favors the formation of linear products. Lowering the reaction pressure increased the reaction efficiency (Table 16; Entry 5).

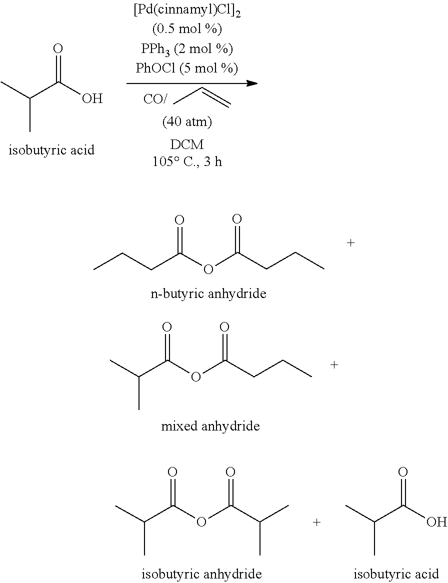

TABLE 16

| | Deviation | | | | | | | | |
|---|---|---|---|---|---|---|---|---|---|
| | from | n-butyric | Mixed | Isobutyric | | | | | |
| | Standard | anhydride | anhydride | anhydride | n-butyric | Isobutyric | Anhydrides | Total | Total |
| Entry | Conditions | (M) | (M) | (M) | acid (M) | acid (M) | (L/B) | L/B | TON |
| 1 | None | 0 | 0.09 | 0.52 | 0.06 | 0.76 | 0.1 | 0.1 | 46 |
| 2 | 22 h | 0 | 0.25 | 0.15 | 0 | 1.16 | 0.4 | 0.1 | 30 |
| | instead of 3 | | | | | | | | |
| | h | | | | | | | | |
| 3 | xantphos | 0.19 | 0.64 | 0.54 | 0.11 | 0.19 | 0.6 | 0.6 | 104 |
| | instead of | | | | | | | | |
| | PPh₃ | | | | | | | | |
| 4 | Dtbpx | 0.09 | 0.34 | 0.32 | 0.15 | 0.30 | 0.5 | 0.5 | 57 |
| | instead of | | | | | | | | |
| | PPh₃ | | | | | | | | |
| 5 | 30 atm | 0 | 0.10 | 0.69 | 0.08 | 1.13 | 0.1 | 0.1 | 60 |
| | instead of | | | | | | | | |
| | 40 atm | | | | | | | | |

Reaction conditions: [Pd(cinnamyl)Cl]₂ (0.5 mol %), ligands (2 mol %), isobutyric acid (1.5 mmol, 1.3M), CO/propylene (40 atm) [p_{propylene} = 8.8 atm, p_{CO} = 31.2 atm], 105° C., 5 mol % benzoyl chloride, 3 h. Molarity was calculated by quantitative ¹³C NMR with HMDSO as an internal standard. TON = (n-butyric anhydride + mixed anhydride + isobutyric anhydride)/mmole of catalyst. Anhydride L/B = ((2*n-butyric anhydride) + mixed anhydride)/(mixed anhydride + (2*isobutyric anhydride)) and total L/B = ((2*n-butyric anhydride) + mixed anhydride + linear acid)/(mixed anhydride + (2*isobutyric anhydride) + isobutyric acid).

### 9. Variation of Starting Substrates

The catalytic reaction can proceed on a variety of types of substrates as shown below.

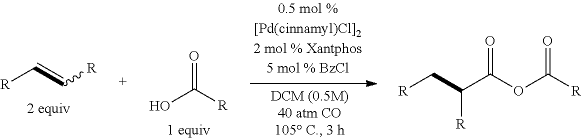

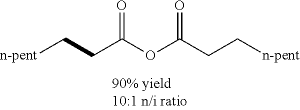

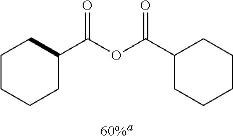

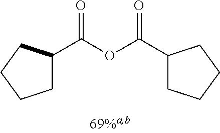

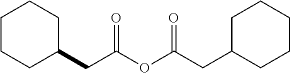

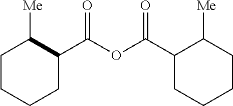

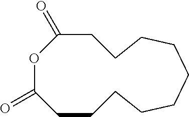

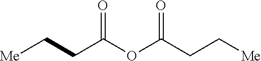

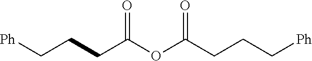

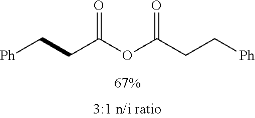

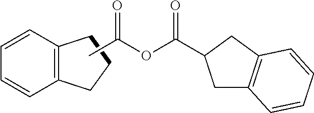

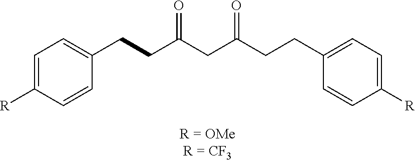

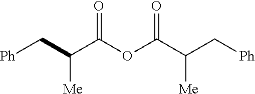

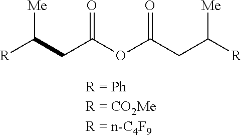

^{a}1 mol % Pd dimer, 4 mol % Xantphos.
^{b}PhCl as solvent.

### 10. Photochemical Carbonylative Anhydride Synthesis

The reactions also proceed using light irradiation. Beginning with a variation of standard hydrocarboxycarbonylation conditions using 0.5 mol % [Pd(cinnamyl)Cl]₂ as a precatalyst with 2 mol % Xantphos ligand and 5 mol % benzoyl chloride as an additive, under 10 atm of CO with irradiation from 370 nm LEDs a combined anhydride yield of 79% was obtained, with an observed 16:1 n/iso ratio, starting with 1-heptene and 3 equiv of octanoic acid (entry 1). Under these conditions, significant amounts of iso-acid were also formed. Using octanoic acid as the limiting reagent prevented this post-catalytic isomerization while there was no change in overall yield of anhydride (entry 2). The use of longer wavelength LEDs (427 nm and 390 nm) resulted in a minor decrease in yield (entries 3 and 4). Benzoyl chloride additive was not necessary for this carbonylation to proceed, as disproportionation of palladium dimers can occur under irradiation (entry 5). Lower yields were observed at lower pressure (1.1 atm CO, entry 6) and shorter reaction time (3 h, entry 7).

Simple palladium precatalyst salts without chloride ligands did not deliver the desired anhydride product, although the active palladium(0) catalyst may not have been formed under these conditions (entries 8 and 9). Other bidentate phosphine ligands with promising results under thermal conditions generated anhydride product in trace yields under photochemical conditions (entries 10-13).

TABLE 17

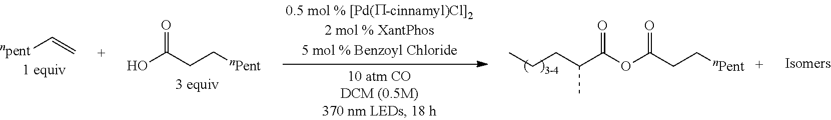

| | | n- | iso- | n- | iso- | Observed |
|---|---|---|---|---|---|---|
| | | Acid | Acid | Anhydride | Anhydride | n/iso |
| Entry | Change in above conditions | (%) ^{a} | (%) ^{a} | (%) ^{a} | (%) ^{a} | ratio |
| 1 | none | 232 | 7.4 | 74 | 4.5 | 16:1 |
| 2 | 2:1 alkene:acid stoichiometry | 15 | <2 | 67 | 13 | 5:1 |
| 3 | 427 nm LEDs instead of 370 nm | 229 | 8.7 | 62 | 2.5 | 25:1 |
| | LEDS | | | | | |
| 4 | 390 nm LEDs instead of 370 nm | 223 | 7.5 | 62 | 3.7 | 17:1 |
| | LEDs | | | | | |
| 5 | no BzCl | 250 | 7.3 | 46 | 3.6 | 13:1 |
| 6 | 1.1 atm CO instead of 10 atm | 244 | 3.9 | 49 | <2 | >20:1 |
| | CO | | | | | |
| 7 | 3 h instead of 18 h | 308 | 4.4 | 20 | <2 | >20:1 |
| 8 | 1 mol % Pd(OAc)₂ instead of | 335 | <2 | <2 | <2 | — |
| | 0.5 mol % [Pd(cinnamyl)Cl]₂ | | | | | |
| 9 | 1 mol % Pd(OAc)₂ instead of | 320 | <2 | 6 | <2 | — |
| | 0.5 mol % [Pd(cinnamyl)Cl]₂, | | | | | |
| | no BzCl | | | | | |
| 10^{b} | DPEphos instead of Xantphos | >99 | <2 | <2 | <2 | — |
| 11^{b} | dppf instead of Xantphos | 68 | <2 | 7.0 | <2 | >20:1 |
| 12^{b} | dtbpx instead of Xantphos | 88 | 2.6 | 6.6 | <2 | >20:1 |
| 13^{b} | dppb instead of Xantphos | 94 | <2 | <2 | <2 | — |

^{a} Yields determined by quantitative ¹³C NMR with HMDSO as an internal standard.
^{b}2:1 alkene:acid stoichiometry.

### 11. Exemplary Batch Catalytic Process (I)

In an argon-filled glovebox, a 2 mL GC vial was charged with a magnetic stir bar, palladium cinnamyl chloride dimer (6.5 mg, 2.5 mol %, 12.5 μmol), xantphos (14.5 mg, 5 mol %, 25 μmol), acetonitrile (1 mL, 0.50 M), octanoic acid (0.24 mL, 3 equiv, 1.50 mmol), and 1-heptene (70 μL, 1 equiv, 0.50 mmol). The vial was capped with a lid containing a pre-cut septum, and loaded into a Parr reactor with any other reactions to be conducted in the same pressure and temperature conditions. The reactor was sealed, removed from the glovebox, pressurized with carbon monoxide (purged 3 times at 10 atm, then pressurized to 40 atm), placed in a pre-heated silicon oil bath, and stirred at 105° C. for 3 hours. Carbon monoxide is toxic, and all manipulations involving CO should be performed in a well-ventilated and functioning fume hood. Personal CO detectors (Draeger Pac 6500 series) were used to monitor the atmosphere during these manipulations. Afterwards, the reactor was removed from the oil bath, allowed to cool to room temperature, and depressurized in a fume hood. A stock solution of tridecane (3 mL, 5.6 mM in DCM) was added to the reaction mixture as an internal standard. The solution was filtered through a 0.45 μm PTFE syringe filter into a 20 mL scintillation vial and quantified by GC or NMR analysis.

GC spectra were obtained using a Shimadzu GC-2010 gas chromatograph with a Shimadzu AOC-20s Autosampler, and Shimadzu SHRXI-5 MS GC column. GC data was obtained using the following method: initial temperature of 30.0° C., ramping at 5.0° C./min until 50.0° C., then ramping at 15.0° C./min until 250.0° C., and holding for 2.0 min. GC yields were supported by comparison to commercially available or independently synthesized products through calibration curves.

Alternatively, the mixture was concentrated under reduced pressure then diluted with CDCl₃ (1 mL), hexamethyldisiloxane (HMDSO, 15 μL) was added as an internal standard, and the mixture was analyzed by NMR. Proton and carbon magnetic resonance spectra (¹H NMR and ¹³C NMR) were recorded on a Bruker Neo 600 with a CryoQNP probe (¹H NMR at 600 MHz and ¹³C at 151 MHz) spectrometer with HMDSO as the internal standard (¹H NMR: HMDSO in CDCl₃ at 0.07 ppm; ¹³C NMR: HMDSO in CDCl₃ at 1.97 ppm). Quantitative ¹³C NMR analysis was obtained using inverse-gated decoupling pulse sequences with long relaxation delays (60 sec D1).

### 12. Exemplary Batch Catalytic Process (II)

All the samples were prepared under nitrogen atmosphere in a glovebox using anhydrous solvents. About 2 mL of the prepared solution including the precursors (1-heptene (0.1M, 28.2 μL), octanoic acid (0.2M, 63.4 μL), and catalyst complex (2:1 L: Pd, 2.5 mM [Pd], 5.0 mM Ligand)) was added to a small glass vial equipped with a magnetic stirrer, septa for splash prevention and a needle, which allows the liquid phase to be exposed to CO while sitting in a stainless-steel autoclave pressurized by CO. The autoclave was sealed and removed from the glovebox, then attached to the gas supply manifold for purging of nitrogen and filling with propylene (if necessary) and carbon monoxide (3×purge at 10 atm, followed by filling to reaction pressure). The autoclave was sealed and disconnected from the manifold after purging manifold CO, then the autoclave was placed in an oil bath and stirred at reaction temperature for 2 hours. After reaction the autoclave was cooled and depressurized and a sample of the reaction mixture was taken for GC analysis (900 μL Solvent+1.8 mM tridecane internal standard and 100 μL of reaction mixture).

### 13. Exemplary Batch Catalytic Process (III)

In an nitrogen-filled glovebox, a 2 mL GC vial was charged with a magnetic stir bar, palladium cinnamyl chloride dimer (3.8 mg, 0.5 mol %, 0.0075 mmol), ligand (2 mol %, 0.03 mmol), a mixture of deuterated and protio dichloromethane (1 mL), butyric acid (0.116 mL, 1.50 mmol) or iso butyric acid (0.136 mL, 1.50 mmol), and benzoyl chloride (8.71 μL, 5%, 0.075 mmol). The vial was capped with a lid containing septum and poked 10 times before loading into a Hel-cat reactor. The reactor was sealed, removed from the glovebox, pressurized with carbon monoxide/propylene (78%/22%) (purged 3 times at 10 atm, then pressurized to desired pressure). The reactor was heated to 105° C. and stirred for desired hours. Carbon monoxide is toxic, and all manipulations involving CO must be performed in a well-ventilated and functioning fume hood. Personal CO detectors (Draeger Pac 6500 series) were used to monitor the atmosphere during these manipulations. Afterwards, the reactor was allowed to cool to room temperature and slowly depressurized in a fume hood.

Hexamethyldisiloxane (HMDSO, 15 μL) was added as an internal standard, transferred into an NMR tube (filtered through a PTFE syringe filter if necessary) and quantified by NMR analysis. Carbon magnetic resonance spectra (¹³C NMR) was recorded on a Bruker Neo 600 with a CryoQNP probe (¹³C at 151 MHz) spectrometer with HMDSO as the internal standard (¹³C NMR: HMDSO in CD₂Cl₂ at 1.97 ppm). Quantitative ¹³C NMR analysis was obtained using inverse-gated decoupling pulse sequences with long relaxation delays (60 sec D1).

### 14. Exemplary Batch Photocatalytic Process

In an argon-filled glovebox, an Ace Glass pressure tube was charged with palladium cinnamyl chloride dimer (1.3 mg, 0.5 mol %, 2.5 μmol), xantphos (5.8 mg, 2 mol %, 10 μmol), DCM (1 mL, 0.50 M), octanoic acid (0.24 mL, 3 equiv, 1.50 mmol), and 1-heptene (70 μL, 1 equiv, 0.50 mmol). The vessel was sealed with a Swagelok connector cap and removed from the glovebox. Inside a fume hood with closed sashes, the tube was pressurized to 5 atm CO, purged 3 times with CO to replace argon, set to 10 atm and stirred for 18 hours under irradiation at 370 nm (Kessil PR160-370). The tube was then depressurized, the reaction mixture diluted with dichloromethane, transferred to a 20 mL scintillation vial, concentrated under reduced pressure, and prepared for NMR analysis in CDCl₃ with HMDSO as an internal standard.

Features and advantages of this disclosure are apparent from the detailed specification, and the claims cover all such features and advantages. Numerous variations will occur to those skilled in the art, and any variations equivalent to those described in this disclosure fall within the scope of this disclosure. Those skilled in the art will appreciate that the conception upon which this disclosure is based may be used as a basis for designing other methods and systems for carrying out the several purposes of this disclosure. As a result, the claims should not be considered as limited by the description or examples.

1. A method for making an organic anhydride comprising contacting an ethylenically unsaturated compound with carbon monoxide and a carboxylic acid in the presence of a catalyst system obtainable by combining palladium or a palladium compound, a phosphine ligand, and a co-catalytic additive.

2. The method of claim 1, wherein the ethylenically unsaturated compound is a monosubstituted, disubstituted, or trisubstituted alkene.

3. The method of claim 1, wherein the ethylenically unsaturated compound is a terminal alkene.

4. The method of claim 1, wherein the ethylenically unsaturated compound has the formula (I): 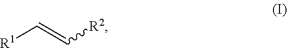
wherein R¹ and R² are independently hydrogen, halide, C₁-C₂₄ alkyl, C₁-C₂₄ heteroalkyl, C₁-C₂₄ alkenyl, C₁-C₂₄ alkynyl, C₁-C₂₄ haloalkyl, C₁-C₂₄ haloalkenyl, cycloalkyl, heterocycloalkyl, bicyclic cycloalkyl, bicyclic heterocycloalkyl, cycloalkenyl, cycloalkynyl, aryl, or heteroaryl; or
wherein R¹ and R² together form a ring having 4 to 10 carbons; and
wherein the wavy bond denotes any geometric isomer.

5. The method of claim 1, which is carried out at a partial pressure of at least 1 atmospheric pressure (atm) of carbon monoxide.

6. (canceled)

7. (canceled)

8. (canceled)

9. The method of claim 1, which is carried out at a temperature of 50° C. to 200° C.

10. (canceled)

11. The method of claim 1, which is carried out in a reactor substantially free of water.

12. The method of claim 1, which is carried out under irradiation from a light source having a wavelength from 300 to 500 nm.

13. (canceled)

14. (canceled)

15. (canceled)

16. The method of claim 1, wherein the carboxylic acid is formed in situ.

17. The method of claim 1, wherein the carboxylic acid has the formula (II): 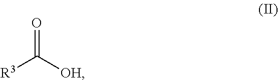
wherein R³ is C₁-C₂₄ alkyl, C₁-C₂₄ alkenyl, C₁-C₂₄ alkynyl, C₁-C₂₄ haloalkyl, C₁-C₂₄ haloalkenyl, C₁-C₂₄ haloalkynyl, cycloalkyl, heterocycloalkyl, bicyclic cycloalkyl, bicycylic heterocycloalkyl, cycloalkenyl, cycloalkynyl, aryl, or heteroaryl.

18. The method of claim 1, wherein the carboxylic acid has one carbon atom more than the ethylenically unsaturated compound.

19. The method of claim 1, wherein the ethylenically unsaturated compound is propylene, the carboxylic acid is isobutyric acid, and the organic anhydride is isobutyric anhydride.

20. (canceled)

21. (canceled)

22. (canceled)

23. (canceled)

24. (canceled)

25. The method of claim 1, wherein the palladium compound is a palladium(0) or palladium(II) compound.

26. The method of claim 1, wherein the palladium compound is tris(dibenzylideneacetone)dipalladium(0), palladium(π-cinnamyl) chloride dimer, Pd(OAc)₂, PdCl₂, Pd(PhCN)₂Cl₂, Pd(MeCN)₂Cl₂, Pd(PPh₃)₂Cl₂, Pd(COD)Cl₂, or [Pd(π-allyl)Cl]₂.

27. The method of claim 1, wherein the phosphine ligand is monodentate or bidentate.

28. The method of claim 1, wherein the phosphine ligand has the formula (III) or (IV): 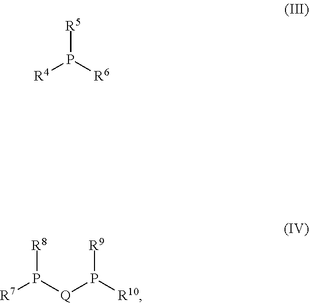
wherein R⁴-R¹⁰ are independently halide, C₁-C₂₄ alkyl, C₂-C₂₄ alkenyl, C₂-C₂₄ alkynyl, C₁-C₂₄ haloalkyl, C₂-C₂₄ haloalkenyl, C₂-C₂₀ haloalkynyl, cycloalkyl, heterocycloalkyl, bicyclic cycloalkyl, bicycylic heterocycloalkyl, cycloalkenyl, cycloalkynyl, aryl, heteroaryl, ferrocenyl, or OR¹¹, wherein R¹¹ is halide, C₁-C₂₄ alkyl, C₂-C₂₄ alkenyl, C₂-C₂₄ alkynyl, C₁-C₂₄ haloalkyl, C₂-C₂₄ haloalkenyl, C₂-C₂₄ haloalkynyl, cycloalkyl, heterocycloalkyl, bicyclic cycloalkyl, bicycylic heterocycloalkyl, cycloalkenyl, cycloalkynyl, aryl, heteroaryl, or ferrocenyl; and
wherein Q is C₁-C₆ alkyl, C₂-C₆ alkenyl, C₂-C₆ alkynyl, aryl, heteroaryl, heteroarenyl, oxydibenzyl, ferrocenyl, quinolinyl, acridinyl, dihydroacridinyl, xanthenyl, or 10H-phenoxazinyl.

29. (canceled)

30. (canceled)

31. The method of claim 1, wherein the phosphine ligand is bis[(2-diphenylphosphino)phenyl]ether (DPEphos) or Xantphos.

32. The method of claim 1, which is carried out neat.

33. (canceled)

34. (canceled)

35. (canceled)

36. (canceled)

37. (canceled)

38. The method of claim 1, wherein the co-catalytic additive is an acid, an organic acid, an acyl electrophile, or a halogenated additive.

39. The method of claim 38, wherein the acid is HCl, the acyl electrophile is trifluoroacetic acid or acetic anhydride, and the halogenated additive is an aryl halide or benzoyl halide.

40. (canceled)

41. (canceled)

42. (canceled)

43. (canceled)

44. (canceled)

45. (canceled)

46. (canceled)

47. (canceled)

48. (canceled)
